(19) United States
(12) Patent Application Publication
(10) Pub. No.: US 2025/0134958 A1
(43) Pub. Date: May 1, 2025

(54) PHARMACEUTICAL FORMULATIONS AND METHODS OF MAKING THE SAME

(71) Applicant: AMGEN INC., Thousand Oaks, CA (US)

(72) Inventors: Monica Goss, Newbury Park, CA (US); Nicole Ball, Thousand Oaks, CA (US)

(21) Appl. No.: 19/010,585

(22) Filed: Jan. 6, 2025

Related U.S. Application Data
(62) Division of application No. 17/933,055, filed on Sep. 16, 2022, now Pat. No. 12,214,013.
Application No. 17/933,055 is a division of application No. 16/144,120, filed on Sep. 27, 2018, now Pat. No. 11,491,223.
Application No. 16/144,120 is a division of application No. 15/958,261, filed on Apr. 20, 2018, now Pat. No. 10,307,483.
Application No. 15/958,261 is a continuation of application No. 15/788,762, filed on Oct. 19, 2017, now abandoned.
(60) Provisional application No. 62/411,458, filed on Oct. 21, 2016.

(51) Int. Cl. A61K 38/17; A61K 9/00; A61K 9/08; A61K 39/395; A61K 47/02; A61K 47/18; A61K 47/26; A61K 47/68; A61P 17/06; A61P 19/02; A61P 29/00; C07K 14/715; C07K 19/00

(52) U.S. Cl. CPC A61K 38/1793; A61K 9/0019; A61K 9/08; A61K 39/39591; A61K 47/02; A61K 47/183; A61K 47/26; A61K 47/68; A61P 17/06; A61P 19/02; A61P 29/00; C07K 14/7151; C07K 19/00; C07K 2319/30; C07K 2319/32

(57) ABSTRACT

The invention relates to the formulation of pharmaceutical compositions of etanercept. The invention also relates to methods of removing buffer and of formulating pharmaceutical compositions of etanercept.

### CROSS-REFERENCE TO RELATED APPLICATION

This application claims the benefit of U.S. Provisional Application No. 62/411,458 filed Oct. 21, 2016, which is incorporated in its entirety by reference herein.

### FIELD OF THE INVENTION

The invention relates to the formulation of pharmaceutical compositions of etanercept. The invention also relates to methods of removing buffer and of formulating pharmaceutical compositions of etanercept.

### BACKGROUND OF THE INVENTION

Formulation of a protein drug can present many challenges for the pharmaceutical scientist. A formulation must be found that stabilizes the protein drug and makes it resistant to degradation by proteolysis, aggregation, misfolding, etc. Especially for engineered proteins that differ in substantial respects to known proteins, finding appropriate stability conditions can be challenging. It is also desirable to have the protein drug be in a format that is convenient for the patient. Desired properties include stability at ambient and refrigerated temperatures; suitability for long term storage, appropriate dosing times and volumes; and minimization of discomfort upon administration.

Etanercept is a dimeric fusion protein consisting of the extracellular ligand-binding portion of the human 75 kilodalton (p75) tumor necrosis factor receptor (TNFR) linked to the Fc portion of human IgG1 The Fc component of etanercept contains the CH2 domain, the CH3 domain and hinge region, but not the CHI domain of IgG1. When expressed in mammalian cells, it forms a homodimeric complex with two domains of the TNF receptor. Thus, it is an artificial protein that is different from both antibodies and soluble TNF receptors, and therefore subject to different degradation pathways than either. Etanercept is commercially available as ENBREL® (Amgen Inc., Thousand Oaks. CA) and is approved to treat moderately to severely active rheumatoid arthritis, moderately to severely active polyarticular juvenile idiopathic arthritis (JIA) in patients ages two and older, chronic moderate to severe plaque psoriasis (PsO) in adults, psoriatic arthritis (PsA) in adults, and active ankylosing spondylitis (AS). Etanercept was first available in a lyophilized formulation to be reconstituted immediately before injection.

Upon reconstitution of the lyophilized product with water for injection, the formulation is 10 mM Tris HCl, 4% mannitol, 1% sucrose, pH 7.4 at about 25 mg/mL. However, this formulation is not stable for storage. It was discovered that a liquid formulation of etanercept could be achieved using arginine to stabilize the protein (see U.S. Pat. No. 7,648,702). An exemplary liquid formulation consists of 50 mg/mL etanercept, 25 mM phosphate buffer, 25 mM L-arginine hydrochloride, 100 mM NaCl, 1% Sucrose at pH 6.3 in water.

### SUMMARY OF THE INVENTION

Provided herein are new and improved formulations of etanercept. In particular, the invention provides pharmaceutical compositions containing etanercept that are stable and can be conveniently stored as a liquid at controlled room temperature (CRT) for extended periods of time, even in the absence of an additional buffering agent. In addition, when the pharmaceutical compositions of the invention are injected into subjects, they also demonstrate significantly reduced injection pain as compared to the commercially available prior art formulation. Thus these pharmaceutical compositions are more convenient and advantageous for patients.

Another aspect of the invention provides methods of formulating pharmaceutical preparations of etanercept at a desired pH but in the absence of additional buffering agent in the final formulation.

In another aspect, the invention provides a pharmaceutical composition comprising etanercept, NaCl, arginine, and sucrose, wherein the pharmaceutical composition has essentially no additional buffering agent, and the pH of the composition is between 6.1 and 6.5. In one embodiment, the pharmaceutical composition is capable of maintaining the pH between 6. 1 and 6.5 when stored at controlled room temperature (CRT) for 2 weeks. In another embodiment, the etanercept concentration is between 40 mg/mL and 100 mg/mL. In another embodiment, the pharmaceutical composition is isotonic. In another embodiment, the pharmaceutical composition contains: between 20 mM and 150 mM NaCl; between 5 mM and 100 mM arginine; and between 0.5% and 2% (w/v) sucrose. In another embodiment, the pharmaceutical composition comprises a surfactant. In another embodiment, the surfactant is polysorbate 20, polysorbate 80, or poloxamer 188. In another embodiment, the surfactant is polysorbate 20 at a concentration (w/v) of between 0.001% and 0.1%. In another embodiment, the surfactant is polysorbate 80 at a concentration (w/v) of between 0.001% and 0.1%. In another embodiment, the surfactant is poloxamer 188 at a concentration (w/v) of between 0.01% to 0.3%. In another embodiment, the pharmaceutical composition maintains a pH of between 5.8 and 6.7 for at least two weeks when stored at approximately 25° C. and wherein less than 6% of the total etanercept is aggregated in a high molecular weight form as assessed using size exclusion chromatography. In another embodiment, the pharmaceutical composition maintains a pH of between about 6.1 and about 6.5. In another embodiment, less than 28% of the total amount of etanercept is in a misfolded form as assessed using hydrophobic interaction chromatography. In another embodiment, the pharmaceutical composition consists essentially of about 40-100 mg/mL etanercept, about 120 mM NaCl, about 25 mM arginine, about 1% sucrose, and water. In another embodiment, the pharmaceutical composition consists essentially of about 40-100 mg/mL etanercept, about 120 mM NaCl, about 25 mM arginine, about 1% sucrose, about 0.01% poly sorbate 20, and water.

In another aspect, the present invention provides a method of formulating a pharmaceutical composition of etanercept to remove an additional buffering agent and maintain pH from 6. 1 to 6.5, comprising formulating the etanercept formulation in a formulation comprising an additional buffering agent at between pH 6.1 and 6.5, and exchanging the formulation comprising an additional buffering agent against a formulation that does not comprise an additional buffering agent and is between pH 5.6 and 6.5, and collecting the resulting pharmaceutical formulation. In one embodiment, the exchange step uses diafiltration. In another embodiment, the formulation that does not comprise an additional buffering agent is isotonic. In another embodiment, the formulation that does not comprise an additional buffering agent contains sucrose, arginine, and NaCl. In another embodiment, the formulation that does not comprise an additional buffering agent contains between 20 mM and 150 mM NaCl; between 5 mM and 100 mM arginine; and between 0.5% and 2% (w/v) sucrose. In another embodiment, the formulation that does not comprise an additional buffering agent consists essentially of about 120 mM NaCl, about 25 mM arginine, about 1% sucrose, and water. In another embodiment, the method of formulating a pharmaceutical composition of etanercept further comprises adding polysorbate. In another embodiment, the polysorbate is polysorbate 20 at a concentration (w/v) of between 0.001% and 0.1%. In another embodiment, the method of formulating a pharmaceutical composition of etanercept further comprises filtering the pharmaceutical composition. In another embodiment, the method of formulating a pharmaceutical composition of etanercept further comprises aliquoting the pharmaceutical composition into a drug product form.

In another aspect, the present invention provides a kit comprising a pharmaceutical composition of etanercept as described above in a drug product form and instructions for storage and use.

In another aspect, the present invention provides a pharmaceutical composition comprising etanercept, NaCl, arginine, sucrose, a phosphate buffer and benzyl alcohol, wherein the pH of the composition is between 6.1 and 6.5. In one embodiment, the benzyl alcohol is at a concentration (v/v) of between 0.1% and 5.0%. In another embodiment, the concentration of benzyl alcohol is about 0.9%. In another embodiment, the pharmaceutical composition comprising etanercept further comprises polysorbate 20 at a concentration (w/v) of between 0.001% and 0.1%. In another embodiment, the concentration of polysorbate 20 is about 0.004% In another embodiment, the pharmaceutical composition comprising etanercept consists essentially of etanercept at about 40-100 mg/mL, arginine at about 25 mM, sodium chloride at about 100 mM, sucrose at a concentration (w/v) of about 1%, phosphate buffer at about 25 mM, and benzyl alcohol at a concentration (v/v) of about 0.9%. In another embodiment, the pharmaceutical composition comprising etanercept consists essentially of etanercept at about 40-100 mg/mL, arginine at about 25 mM, sodium chloride at about 100 mM, sucrose at a concentration (w/v) of about 1%, phosphate buffer at about 25 mM, benzyl alcohol at a concentration (v/v) of about 0.9%, and polysorbate 20 at a concentration (w/v) of about 0.004%.

In another aspect, the present invention provides a single-dose container containing a pharmaceutical composition comprising etanercept as described above. In one embodiment, the pharmaceutical composition consists essentially of etanercept at about 40-100 mg/mL, arginine at about 25 mM, sodium chloride at about 100 mM, sucrose at a concentration (w/v) of about 1%, phosphate buffer at about 25 mM, and benzyl alcohol at a concentration (v/v) of about 0.9%. In another embodiment, the pharmaceutical composition consists essentially of etanercept at about 40-100 mg/mL, arginine at about 25 mM, sodium chloride at about 100 mM, sucrose at a concentration (w/v) of about 1%, phosphate buffer at about 25 mM, benzyl alcohol at a concentration (v/v) of about 0.9%, and polysorbate 20 at a concentration (w/v) of about 0.004%. In another embodiment, the single dose container is a vial, a syringe, or an autoinjector. In another embodiment, the single-dose container contains an aqueous formulation consisting of etanercept at 50.0 mg/mL, sodium chloride at 120 mM, L-arginine at 25 mM, sucrose at 1.0% (w/v).

In another aspect, the present invention provides a method of preparing a single-dose container containing a pharmaceutical composition comprising etanercept as described above, comprising filling the single-dose container with about a single dose of the pharmaceutical composition under sterile conditions.

### BRIEF DESCRIPTION OF THE DRAWINGS

FIG. 1 shows the percent HMW (peak B) as detected by SEC for the etanercept stability assay of Example 3.

FIG. 2 shows the percent LMW as detected by dSEC for the etanercept stability assay of Example 3.

FIG. 3 shows the percent Peak 3 as detected by HIC for the etanercept stability assay of Example 3.

FIG. 4 shows the percent Peak 3 as detected by HIC for the stainless steel cryo-vessel storage etanercept stability assay of Example 4.

FIG. 5 shows the percent LMW as detected by dSEC for the stainless steel cryo-vessel storage etanercept stability assay of Example 4.

FIG. 6 shows the percent Peak B as detected by SEC for the stainless steel cryo-vessel storage etanercept stability assay of Example 4.

FIG. 7 shows the percent Peak B as detected by SEC for the freeze-thaw etanercept stability assay of Example 4.

FIG. 8 shows the pH stability of the conditioned AEX intermediate pool at controlled room temperature (CRT) in the assay of Example 6.

FIG. 9 shows the UF/DF pool pH (A) and conductivity (B) stability at CRT in the assay of Example 6.

FIG. 10 shows the pH (A) and conductivity (B) stability of etanercept formulated in SAS solution in the assay of Example 6.

### DETAILED DESCRIPTION OF THE INVENTION

The invention provides improved pharmaceutical compositions of etanercept. As used herein, the phrase “pharmaceutical composition” is understood to refer to a formulation of a polypeptide suitable for injection and/or administration into a patient in need thereof. More particularly, a pharmaceutical composition is substantially sterile and does not contain any agents that are unduly toxic or infectious to the recipient. Etanercept is a soluble form of the p75 TNF receptor fused to an Fc domain of a human IgG1 (TNFR:Fc). A commercially available etanercept is known as ENBREL® (Immunex Inc., Thousand Oaks. CA). Etanercept is produced by recombinant DNA technology in a Chinese hamster ovary (CHO) mammalian cell expression system. It consists of 934 amino acids and has an apparent molecular weight of approximately 150 kilodaltons (Physicians Desk Reference, 2002, Medical Economics Company Inc.). The full sequence expressed in CHO cells is shown below. However, it is to be understood that minor modifications and deletions of this sequence (up to 10%) may be possible and can be used within the scope of the invention.

| | |
|---|---|
| 1 | Leu-Pro-Ala-Gln-Val-Ala-Phe-Thr-Pro-Tyr- |
| 11 | Ala-Pro-Glu-Pro-Gly-Ser-Thr-Cys-Arg-Leu- |
| 21 | Arg-Glu-Tyr-Tyr-Asp-Gln-Thr-Ala-Gln-Met- |
| 31 | Cys-Cys-Ser-Lys-Cys-Ser-Pro-Gly-Gln-His- |
| 41 | Ala-Lys-Val-Phe-Cys-Thr-Lys-Thr-Ser-Asp- |
| 51 | Thr-Val-Cys-Asp-Ser-Cys-Glu-Asp-Ser-Thr- |
| 61 | Tyr-Thr-Gln-Leu-Trp-Asn-Trp-Val-Pro-Glu- |
| 71 | Cys-Leu-Ser-Cys-Gly-Ser-Arg-Cys-Ser-Ser- |
| 81 | Asp-Gln-Val-Glu-Thr-Gln-Ala-Cys-Thr-Arg- |
| 91 | Glu-Gln-Asn-Arg-Ile-Cys-Thr-Cys-Arg-Pro- |
| 101 | Gly-Trp-Tyr-Cys-Ala-Leu-Ser-Lys-Gln-Glu- |
| 111 | Gly-Cys-Arg-Leu-Cys-Ala-Pro-Leu-Arg-Lys- |
| 121 | Cys-Arg-Pro-Gly-Phe-Gly-Val-Ala-Arg-Pro- |
| 131 | Gly-Thr-Glu-Thr-Ser-Asp-Val-Val-Cys-Lys- |
| 141 | Pro-Cys-Ala-Pro-Gly-Thr-Phe-Ser-Asn-Thr- |
| 151 | Thr-Ser-Ser-Thr-Asp-Ile-Cys-Arg-Pro-His- |
| 161 | Gln-Ile-Cys-Asn-Val-Val-Ala-Ile-Pro-Gly- |
| 171 | Asn-Ala-Ser-Met-Asp-Ala-Val-Cys-Thr-Ser- |
| 181 | Thr-Ser-Pro-Thr-Arg-Ser-Met-Ala-Pro-Gly- |
| 191 | Ala-Val-His-Leu-Pro-Gln-Pro-Val-Ser-Thr- |
| 201 | Arg-Ser-Gln-His-Thr-Gln-Pro-Thr-Pro-G!u- |
| 211 | Pro-Ser-Thr-Ala-Pro-Ser-Thr-Ser-Phe-Leu- |
| 221 | Leu-Pro-Met-Gly-Pro-Ser-Pro-Pro-Ala-Glu- |
| 231 | Gly-Ser-Thr-Gly-Asp-Glu-Pro-Lys-Ser-Cys- |
| 241 | Asp-Lys-Thr-His-Thr-Cys-Pro-Pro-Cys-Pro- |
| 251 | Ala-Pro-Glu-Leu-Leu-Gly-Gly-Pro-Ser-Val- |
| 261 | Phe-Leu-Phe-Pro-Pro-Lys-Pro-Lys-Asp-Thr- |
| 271 | Leu-Met-Ile-Ser-Arg-Thr-Pro-Glu-Val-Thr- |
| 281 | Cys-Val-Val-Val-Asp-Val-Ser-His-Glu-Asp- |
| 291 | Pro-Glu-Val-Lys-Phe-Asn-Trp-Tyr-Val-Asp- |
| 301 | Gly-Val-Glu-Val-His-Asn-Ala-Lys-Thr-Lys- |
| 311 | Pro-Arg-Glu-Glu-Gln-Tyr-Asn-Ser-Thr-Tyr- |
| 321 | Arg-Val-Val-Ser-Val-Leu-Thr-Val-Leu-His- |
| 331 | Gln-Asp-Trp-Leu-Asn-Gly-Lys-Glu-Tyr-Lys- |
| 341 | Cys-Lys-Val-Ser-Asn-Lys-Ala-Leu-Pro-Ala- |
| 351 | Pro-Ile-Glu-Lys-Thr-Ile-Ser-Lys-Ala-Lys- |
| 361 | Gly-Gln-Pro-Arg-Glu-Pro-Gln-Val-Tyr-Thr- |
| 371 | Leu-Pro-Pro-Ser-Arg-Glu-Glu-Met-Thr-Lys- |
| 381 | Asn-Gln-Val-Ser-Leu-Thr-Cys-Leu-Val-Lys- |
| 391 | Gly-Phe-Tyr-Pro-Ser-Asp-Ile-Ala-Val-Glu- |
| 401 | Trp-Glu-Ser-Asn-Gly-Gln-Pro-Glu-Asn-Asn- |
| 411 | Tyr-Lys-Thr-Thr-Pro-Pro-Val-Leu-Asp-Ser- |
| 421 | Asp-Gly-Ser-Phe-Phe-Leu-Tyr-Ser-Lys-Leu- |
| 431 | Thr-Val-Asp-Lys-Ser-Arg-Trp-Gln-Gln-Gly- |
| 441 | Asn-Val-Phe-Ser-Cys-Ser-Val-Met-His-Glu- |
| 451 | Ala-Leu-His-Asn-His-Tyr-Thr-Gln-Lys-Ser- |
| 461 | Leu-Ser-Leu-Ser-Pro-Gly-Lys |

The invention provides a pharmaceutical composition comprising etanercept but containing essentially no additional buffering agent. The phrase “additional buffering agent” refers to a component of an etanercept composition or formulation, other than etanercept itself, that contributes significantly to the buffering capacity of the composition or formulation. Etanercept itself has been shown herein to provide all the needed buffering to maintain the pH between 6.1 and 6.5, and in particular at about 6.2-6.3, under the conditions described below. As demonstrated below in Example 1, this pH range has been shown to be effective to maintain the desired stability characteristics of an etanercept formulation (less than 6% high molecule weight aggregates and less than 28% misfolded and clipped species).

The phrase “essentially no additional buffering agent” means that there is less than 0.5 mM of any buffering agent other than etanercept. The phrase “total additional buffering agent” refers collectively to all of the components of an etanercept composition or formulation, except for etanercept itself, that contribute significantly to the buffering capacity of the composition or formulation. In certain embodiments, pharmaceutical compositions according to the invention comprise less than 2.0 mM total additional buffering agent, less than 1.5 mM total additional buffering agent, less than 1.0 mM total additional buffering agent, less than 0.5 mM total additional buffering agent, less than 0.25 mM total additional buffering agent, less than 0.1 mM total additional buffering agent, or less than 0.05 mM of total additional buffering agent. In typical pharmaceutical compositions, additional buffering agents are used to maintain the pH in a desired range, often at concentrations of 5.0 mM or higher. Various well known additional buffering agents are histidine, potassium phosphate, sodium or potassium citrate, maleic acid, ammonium acetate, tris-(hydroxymethyl)-aminomethane (tris), various forms of acetate and diethanolamine. One common buffering agent is sodium phosphate as its buffering capacity is at or near pH 6.2. Sodium phosphate is the buffering agent used in the current commercial liquid formulation of etanercept since its desired pH is 6.3. In the invention described herein, essentially no sodium phosphate is present in the pharmaceutical formulation of etanercept. Surprisingly, despite the absence of essentially any additional buffering agent, the pH of the inventive pharmaceutical composition is maintained between 6.1 and 6.5, even after extended storage. Even more surprisingly, when injected into subjects (e.g., a human subject or a patient), the pharmaceutical composition with essentially no additional buffering agent results in significantly less pain than the current buffered commercial formulation. Although phosphate is often chosen as a buffer for pharmaceutical compositions because of the close to neutral pH buffering capacity and belief that it is one of the less painful buffer components (as compared to, for example, citrate buffer), the instant inventors have determined that phosphate buffer at around pH 6.3 does contribute to pain upon injection.

Unless otherwise clear from the context of its use, a “formulation solution” or “formulation buffer” is a solution or buffer that does not itself contain etanercept but is used to make a formulation comprising etanercept.

Typically, the etanercept concentration in the pharmaceutical compositions of the invention is between about 40 mg/mL and about 200 mg/mL in an aqueous formulation (e.g., water as the solvent). More preferably, the etanercept concentration is between about 40 mg/mL and about 100 mg/mL, vet more preferably between about 40 mg/mL and about 75 mg/mL, and optionally about 50 mg/mL.

The pharmaceutical compositions of the invention also contain arginine. Arginine has been shown to make a substantial contribution to stabilizing etanercept in a liquid formulation (see U.S. Pat. No. 7,648,702, incorporated herein by reference). Pharmaceutically appropriate forms of arginine are commercially available. Typically, L-arginine (e.g., L-arginine HCl or L-arginine base) is the arginine used for pharmaceutical formulations. It is understood that within the pH range of 6.0 and 6.6, and in particular at a pH of about 6.2-6.3, arginine does not contribute meaningfully to the buffering capacity of a formulation. Accordingly, it is not an additional buffering agent in the etanercept formulations or compositions of the invention. The concentration of arginine in the compositions of the invention are preferably from about 1 mM to about 1 M, more preferably from about 10 mM to about 200 mM, or alternatively from about 5 mM to about 100 mM, more preferably from about 10 mM to about 100 mM, even more preferably from about 15 mM to about 75 mM, and yet more preferably at about 25 mM. Thus, in one aspect of the invention, the pharmaceutical composition comprises about 50 mg/mL to 75 mg/mL etanercept and about 25 mM arginine, wherein the pharmaceutical composition has essentially no additional buffering agent, and the pH of the composition is between 6.0 and 6.6. As used herein, the term “about” is understood to mean that there can be variation in the concentration of a component of the described formulation that can be up to and including 10% of the given value. For example, if a formulation has about 10 mg/mL of a polypeptide, this is understood to mean that a formulation can have between 9 to 11 mg/mL, of the stated polypeptide.

The pharmaceutical compositions may contain additional excipients, as long as those excipients are not additional buffering agents, and in particular are not phosphate buffering agents. Examples of additional excipients according to the invention include but are not limited to sugars/polyols such as: sucrose, lactose, glycerol, xylitol, sorbitol, mannitol, maltose, inositol, trehalose, glucose; polymers such as: serum albumin (bovine serum albumin (BSA), human SA or recombinant HA), dextran, PVA, hydroxypropyl methylcellulose (HPMC), polyethyleneimine, gelatin, polyvinylpyrrolidone (PVP), hydroxyethylcellulose (HEC); non-aqueous solvents such as: polyhydric alcohols, (e.g., PEG, ethylene glycol and glycerol) dimethysulfoxide (DMSO) and dimethylformamide (DMF); amino acids such as: proline, L-serine, alanine, glycine, lysine hydrochloride, sarcosine and gamma-aminobutyric acid, and surfactants.

In some preferred embodiments of the invention, the excipients include NaCl and/or sucrose. NaCl may be present in the pharmaceutical composition at a concentration of from about 5 mM to about 200 mM, more preferably between about 20 mM to about 150 mM, even more preferably between about 80 mM to about 140 mM. Sucrose may be added to a concentration of between about 0.5% to about 2% (w/v) sucrose, more preferably between about 0.8% to about 1.2% (w/v) sucrose, even more preferably at about 1% (w/v) sucrose.

The osmolality of a pharmaceutical composition is preferably regulated in order to maximize the active ingredient's stability and also to minimize discomfort to the patient upon administration. It is generally preferred that a pharmaceutical composition be isotonic with serum, i.e., having the same or similar osmolality, which is achieved by addition of a tonicity modifier. Serum is approximately 300 +/−50 milliosmolals per kilogram, thus it is contemplated that the osmolality of an isotonic pharmaceutical composition will be from about 180 to about 420 milliosmolals. In some embodiments, the range will be from about 250 to about 350 milliosmolals.

A tonicity modifier is understood to be a molecule that contributes to the osmolality of a solution. Examples of tonicity modifiers suitable for modifying osmolality include, but are not limited to amino acids (e.g., arginine, cysteine, histidine and glycine), salts (e.g., sodium chloride, potassium chloride and sodium citrate) and/or saccharides (e.g., sucrose, glucose and mannitol). The concentration of the tonicity modifier in the formulation is preferably between about 1 mM to 1 M, more preferably about 10 mM to about 200 mM. In some embodiments, the concentrations of NaCl and sucrose are adjusted to generate a pharmaceutical composition that is isotonic. In some embodiments illustrated below by way of example, the pharmaceutical composition contains about 40-100 mg/mL etanercept, about 120 mM NaCl, about 25 mM arginine, about 1% sucrose, and water. In particular, the pharmaceutical composition can consist essentially of about 50-100 mg/mL, etanercept, about 120 mM NaCl, about 25 mM arginine, about 1% sucrose, about 0.01% polysorbate 20, and water.

Optionally, the pharmaceutical compositions of the invention may include a surfactant. Surfactants are agents that reduce solution/surface induced stress. Examples of surfactants are polysorbates, such as polysorbate 20, polysorbate 40, polysorbate 60, and polysorbate 80 (e.g., TWEEN-20R (Sigma-Aldrich, St. Louis, MO) or TWEEN-80® (Sigma-Aldrich, St. Louis, MO)), sodium dodecyl sulfate (SDS), polyoxyethylene copolymer, poloxamers, such as poloxomer 188 (e.g., PLURONIC R F-68 (Sigma-Aldrich, St. Louis, MO) or poloxomer 407 (e.g., PLURONIC® F-127 (Sigma-Aldrich, St. Louis, MO), CHAPS, monolaurate, or any combination of the above. A preferred surfactant is polysorbate 20. For example, polysorbate 20 can be included in the pharmaceutical compositions at a concentration (w/v) of between about 0.001% and about 0.03%. In particular embodiments illustrated below by example, polysorbate 20 can be included in the pharmaceutical formulations at a concentration (w/v) of 0.01% or at about 0.004%.

### Testing of the Pharmaceutical Compositions

The examples below illustrate how one of skill in art can determine whether a formulation is capable of maintaining the pH in a desired range Essentially, the pharmaceutical composition is formulated and stored in the test containers (which may be glass vials, glass syringes, plastic syringes, stainless steel vessels, or any manner of sterile device suitable for pharmaceutical compositions) and the pH is assessed at time 0, and then at indicated times as appropriate. Usually, the testing conditions will anticipate needs for storage of the pharmaceutical composition, and will stress those conditions. For example, the formulations of the invention are able to maintain the desired pH under controlled room temperature (CRT) for at least 2 weeks, at least 4 weeks, at least 8 weeks, at least 12 weeks, and at least 24 weeks. CRT is defined by the USP, and has a temperature maintained thermostatically that encompasses the usual and customary working environment of 20° C. to 25° C. (68° F. to 77° F.); that results in a mean kinetic temperature calculated to be not more than 25° C.; and that allows for excursions between 15° C. and 30° C. (59° F. and 86° F.) that are experienced in pharmacies, hospitals, and warehouses.

In one aspect, the pharmaceutical compositions of the invention exhibit particular quality attributes. The testing of these quality attributes is also described below by way of example. For example, the pharmaceutical compositions of the invention contain less than 6% of the total etanercept aggregated in a high molecular weight form as assessed using size exclusion chromatography. As another example, the pharmaceutical compositions of the invention contain less than 28% of the total amount of etanercept is in a misfolded form as assessed using hydrophobic interaction chromatography.

The pharmaceutical compositions of the invention are capable of remaining stable by maintaining pH and/or other noted quality attributes (minimum high molecular weight forms and minimum misfolded forms) for the following temperatures and extended time periods (1) at −30° C. (frozen) for at least 4 weeks, at least 3 months, at least 6 months, at least 12 months, and at least 36 months; (2) for up to 1 freeze/thaw cycle, up to 2 freeze/thaw cycles, up to 3 freeze/thaw cycles, and up to 5 freeze/thaw cycles; (3) at 4° C. (refrigerated temperature) for at least 2 weeks, at least 4 weeks, at least 8 weeks, at least 12 weeks, at least at least 24 weeks, and at least 52 weeks; (4) at 25° C. (room temperature) for at least 2 weeks, at least 4 weeks, at least 8 weeks, at least 12 weeks, at least 24 weeks; and (5) at 40° C. (accelerated stability testing) for at least 2 weeks.

The pharmaceutical compositions of the invention also exhibit the surprising result of reduced pain upon injection into a subject. This property can be assessed using the Visual Analog Scale (VAS) that has been validated by Gallagher et al, 2002, Am. J. Em. Med. v20; i4: 287-290. Trained health professionals administer the drug via injection, and within 30 seconds after each injection, subjects assessed their level of injection pain using a 100 mm Visual Analog Scale (VAS). A difference of 13 to 16 mm on the VAS is considered to be clinically meaningful. Using this technique, it was demonstrated that a placebo formulation without phosphate induced significantly less pain than a placebo formulation containing phosphate at pH 6.3 and the current commercial formulation containing both etanercept and phosphate at pH 6.3.

Production and purification of the etanercept to be used in the pharmaceutical compositions and methods of the invention can be performed by any standard method. Typically, etanercept is expressed recombinantly in CHO cells and secreted into the medium. The medium is collected, filtered, and purified using, for example, various chromatography techniques. For example, protein A can be used to purify Fc domain containing polypeptides such as etanercept, and is advantageous as a first processing step. Other techniques for polypeptide purification such as fractionation on an ion-exchange column, ethanol precipitation, reverse phase HPLC, chromatography on silica, chromatography on heparin SEPHAROSET™, chromatography on an anion or cation exchange resin (such as a polyaspartic acid column), hydroxyapatite chromatography, gel electrophoresis, dialysis, and affinity chromatography, and any combination of purification techniques known or yet to discovered. Examples of useful production and purification techniques can be found in U.S. Pat. No. 7,294,481 (Fung), U.S. Pat. No. 7,452,695 (Van Ness et al.), U.S. Pat. No. 7,122,641 (Vedantham et al.), U.S. Pat. No. 7,157,557 (Sassenfeld et al.), U.S. Pat. No. 7,300,773 (Drapeau et al.), U.S. Pat. No. 8,163,522 (Brockhaus et al.), and U.S. Pat. No. 7,648,702 (Gombotz et al.).

### Methods of the Invention

The invention also provides a method of formulating a pharmaceutical composition of etanercept to remove buffer and maintain pH at a target range, comprising formulating the etanercept in a buffered formulation in the target range, and exchanging the buffered formulation against an unbuffered formulation that is within in or just below that target range, and collecting the resulting pharmaceutical formulation of the etanercept. In a preferred embodiment illustrated below by way of example, the method provides formulating a pharmaceutical composition of etanercept to remove buffer and maintain pH from 6.0 to 6.6, comprising formulating the etanercept formulation in a buffered formulation at between pH 6.0 to 6.6, and exchanging the buffered formulation against an unbuffered formulation that is between pH 5.6 and 6.5, and collecting the resulting pharmaceutical formulation. In order to achieve an unbuffered composition of etanercept that maintains its pH from 6.1 to 6.5, it is important to ensure that the pH of both the starting buffered etanercept formulation and the unbuffered formulation is calibrated. For example, if the starting buffered etanercept formulation is at pH 7.2, it will be adjusted with a strong acid, such as HCl, to within the range 6.1 to 6.5. Similarly, the unbuffered formulation that is used for exchange should be titrated to between pH 5.6 and 6.5. Because the unbuffered formulation used for exchange has no buffering agent, care should be used during titration.

To exchange the buffered formulation against an unbuffered formulation, one of skill in the art can make use of a variety of buffer exchange techniques that are well known in the art. Dialysis makes use of selective diffusion through a semi-permeable membrane to remove unwanted smaller molecules from a larger protein formulation. In one embodiment, equilibrations are done serially until a desired fold reduction in the concentration of an unwanted molecule is achieved. For example, three serial equilibrations, each at or greater than 100-fold dilution, can be used to achieve a concentration reduction of 1,000,000-fold or greater. Ultrafiltration and diafiltration are similar to dialysis in that they use a semi-permeable membrane. But unlike the passive diffusion of dialysis, ultrafiltration and diafiltration involves forcing solutions through the membrane using various techniques. Pressure and centrifugation are typically used. Still another method of buffer exchange can be performed using gel filtration or size exclusion chromatography. There are many other chromatographic techniques that also can be used to achieve buffer exchange that are well within the skill of those in the art such as ion exchange chromatography, hydrophobic interaction chromatography, and mixed mode chromatography

After the buffered formulation is exchanged into the unbuffered formulation, the methods of the invention include collecting the resulting pharmaceutical formulation. At this point, essentially all buffer has been removed, but the pH is still maintained at the desired levels. For a pharmaceutical composition containing etanercept, the pH is maintained at between 6.0 and 6.6.

The pharmaceutical formulation may be further treated as necessary. For example, a surfactant can be added. In another example, if desired to remove particles, the pharmaceutical composition can be filtered. Alternatively or in addition, the methods of the invention also include aliquoting the pharmaceutical compositions into a drug product form. Such drug product forms are distributed for final use by patients or health care providers. The pharmaceutical compositions of this invention are particularly useful for parenteral administration, i.e., subcutaneously, intramuscularly, intravenously, intraperitoneal, intracerebrospinal, intra-articular, intrasynovial, and/or intrathecal. Parenteral administration can be by bolus injection or continuous infusion. Pharmaceutical compositions for injection may be presented in unit dosage form, e.g., in ampoules or in multi-dose containers. The pharmaceutical compositions may, if desired, be presented in a vial, pack or dispenser device which may contain one or more unit dosage forms containing the active ingredient. In one embodiment the dispenser device can comprise a syringe having a single dose of the liquid formulation ready for injection. In another embodiment, the pharmaceutical composition is aliquoted into a cassette component for use with a reusable autoinjector. Yet another aspect of the invention, the pharmaceutical compositions can be provided packaged in or with an on-body injector device. In still another embodiment, the pharmaceutical compositions can be aliquoted into a drug product form suitable for a needleless injection device.

The pharmaceutical composition can also be aliquoted into a format suitable as a depot preparation. Such long acting formulations may be administered by implantation (for example subcutaneously or intramuscularly) or by intramuscular injection. Thus, for example, the formulations may be modified with suitable polymeric or hydrophobic materials (for example as an emulsion in an acceptable oil) or ion exchange resins, or as sparingly soluble derivatives, for example, as a sparingly soluble salt.

In another embodiment, the present invention is directed to a kit or container, which contains the pharmaceutical composition of the invention. The kit can also be accompanied by instructions for the storage and use of the pharmaceutical compositions. The container can be, for example, a single-use container, i.e., a container that holds one dose formulation of the present invention. It is understood that a single-use container might contain a single dose plus enough extra to ensure that a full single dose can be administered to a patient from the container, but not so much extra that the container could be used to administer a second dose. Examples of containers suitable for use in certain aspects of the present invention (whether they be single-use or multiple-use containers) include vials, syringes, and auto-injectors. Examples of suitable auto-injectors include those found in U.S. Pat. Nos. 8,177,749, 8,052,645, and 8,920,374, in U.S. patents application Ser. Nos. 12/993,163, 13/269750, 13/454531, 14/112479, 14/777255, and 14/777259, and in PCT Publications WO 2014/0089393, WO 2016/033496, and WO 2016/033507, each of which is incorporated herein by reference in its entirety.

The etanercept-containing compositions and formulations of the present invention, as well as the syringes, autoinjectors, kits, and the like described herein, can be used in the treatment of patients with conditions that respond to treatment with etanercept. Examples of such conditions include rheumatoid arthritis, psoriatic arthritis, ankylosing spondylitis, and psoriasis. Methods of treating patients with etanercept are described in, for example, U.S. Pat. Nos. 7,915,225, 8,119,605, 8,410,060, 8,722,631, and 8,119,604, each of which is incorporated herein by reference in its entirety.

The invention will be more fully understood by reference to the following examples. The examples should not, however, be construed as limiting the scope of the invention.

### EXAMPLES

### Example 1: Stability Testing of Various Formulations

This example demonstrates the effects of pH and buffer on etanercept at 50 mg/mL, and assesses the stability of a high concentration (100 mg/mL) solution without added phosphate buffer. The following formulations were tested.

TABLE 1
Formulations for pH Screen

| | | | | | Protein |
|---|---|---|---|---|---|
| | | | Polysorbate | Final | Conc |
| Formulation Name | Buffer | Excipients | Added | pH | (mg/mL) |
| A45SuT | 10 mM | 9% sucrose | 0.004% | 4.5 | 50 |
| | sodium | | PS20 | | |
| | acetate | | | | |
| A52SuT | 10 mM | 9% sucrose | 0.004% | 5.2 | 50 |
| | sodium | | PS20 | | |
| | acetate | | | | |
| A58SuT | 10 mM | 9% sucrose | 0.004% | 5.8 | 50 |
| | sodium | | PS20 | | |
| | acetate | | | | |
| 50_SAST_100NaCl | None | 25 mM L- | 0.004% | 6.3 | 50 |
| | | arginine, 100 mM | PS20 | | |
| | | NaCl, 1% sucrose | | | |
| 100_SAST_100NaCl | None | 25 mM L- | 0.004% | 6.3 | 100 |
| | | arginine, 100 mM | PS20 | | |
| | | NaCl, 1% sucrose | | | |
| PASST + BeOH | 25 mM | 25 mM L- | 0.004% | 6.3 | 50 |
| | phosphate | arginine, 100 mM | PS20 | | |
| | | NaCl, 1% sucrose, | | | |
| | | 0.9% benzyl alcohol | | | |
| PASST (control) | 25 mM | 25 mM L- | 0.004% | 6.3 | 50 |
| | phosphate | arginine, 100 mM | PS20 | | |
| | | NaCl, 1% sucrose | | | |

Materials: Enbrel drug substance in PASS (25 mM phosphate buffer, 25 mM L-arginine, 100 mM NaCl, 1% sucrose) at 50 mg/mL was used for this study. For the acetate and buffer-free formulation, the material was dialyzed into the new formulations (without polysorbate) and concentrated to 50 mg/mL using 10,000 MWCO centripreps. A sample of 50_SAS_100NaCl was also concentrated to 100 mg/mL (100_SAS_100NaCl). Benzyl alcohol was spiked into the current formulation to a final concentration of 0.9%. A 1% stock solution of polysorbate 20 was prepared fresh and spiked into all formulation to a final concentration of 0.004%. All formulations were manually filled into 1 mL long BD glass syringes to a volume of 0.5 mL and then stoppered using an ASPU vacuuming stoppering unit.

Methods: The pH was measured using a Mettler Toledo SevenEasy pH meter combined with a Mettler Inlab MicroProbe. Samples were warmed to room temperature prior to measurements. Osmolality was measured using The Advanced Osmometer Model 3900. Each measurement was performed using 250 μL of sample and 290 osmolality standards were tested to ensure the system was operating properly. Size exclusion HPLC was run on an Agilent 1100 HPLC with Chromeleon 7.2 software. Denatured size exclusion HPLC was run on an Agilent 1100 HPLC with Chromeleon 7.2 software.

Results: The pH for all formulations was maintained over 24 weeks.

TABLE 2
pH at indicated time points and temperatures

| | pH | | | |
|---|---|---|---|---|
| | t = 0 | t = 24 w | | |
| Sample | 4° C. | 4° C. | 25° C. | 40° C. |
| A45SuT | 4.60 | 4.66 | 4.66 | 4.61 |
| A52SuT | 5.10 | 5.13 | 5.16 | 5.08 |
| A58SuT | 5.61 | 5.60 | 5.66 | 5.62 |
| 50_SAST_100NaCl | 6.24 | 6.21 | 6.22 | 6.17 |
| 100_SAST_100NaCl | 6.32 | 6.21 | 6.20 | 6.19 |
| PASST + BeOH | 6.27 | 6.22 | 6.22 | 6.21 |
| PASST control | 6.26 | 6.21 | 6.21 | 6.19 |

TABLE 3
Aggregate levels (Peak B) by SEC, % of total, 4° C.

| sample | t = 0 | t = 4 w | t = 8 w | t = 12 w | t = 24 w |
|---|---|---|---|---|---|
| A45SuT | 0.9 | 1.1 | 1.1 | 1.2 | 1.2 |
| A52SuT | 1.2 | 1.4 | 1.5 | 1.6 | 1.4 |
| A58SuT | 1.0 | 1.2 | 1.2 | 1.3 | 1.3 |
| 50_SAST_100NaCl | 1.1 | 1.3 | 1.3 | 1.3 | 1.4 |
| 100_SAST_100NaCl | 1.1 | 1.4 | 1.5 | 1.5 | 1.6 |
| PASST + BeOH | 1.0 | 1.2 | 1.2 | 1.3 | 1.3 |
| PASST control | 1.0 | 1.1 | 1.2 | 1.2 | 1.3 |

TABLE 4
Aggregate levels (Peak B) by SEC, % of total, 25° C.

| sample | t = 0 | t = 4 w | t = 8 w | t = 12 w | t = 24 w |
|---|---|---|---|---|---|
| A45SuT | 0.9 | 2.0 | 2.7 | 3,2 | 4.1 |
| A52SuT | 1.2 | 2.7 | 3.1 | 3.6 | 4.6 |
| A58SuT | 1.0 | 1.9 | 2.6 | 3.1 | 4.4 |
| 50_SAST_100NaCl | 1.1 | 1.9 | 2.4 | 2.8 | 3.7 |
| 100_SAST_100NaCl | 1.1 | 2.6 | 3.3 | 3 9 | 5.3 |
| PASST + BeOH | 1.0 | 1.9 | 2.4 | 2.8 | 3.8 |
| PASST control | 1.0 | 1.8 | 2.2 | 2.6 | 3.5 |

TABLE 5
Aggregate levels (Peak B) by SEC, % of total, 40° C.

| | t = | t = | t = | t = | t = | t = |
|---|---|---|---|---|---|---|
| sample | 0 | 2 w | 4 w | 8 w | 12 w | 24 w |
| A45SuT | 0.9 | 5.6 | 9.0 | 11.9 | 11.9 | 9.6 |
| A52SuT | 1.2 | 6.3 | 12.9 | 13.6 | 16.4 | 17.9 |
| A58SuT | 1.0 | 5.1 | 9.1 | 14.7 | 18.4 | 24.5 |
| 50_SAST_100NaCl | 1.1 | 3.9 | 6.8 | 12.1 | 16.0 | 26.0 |
| 100_SAST_100NaCl | 1.1 | 5.9 | 10.8 | 18.5 | 24.0 | 37.8 |
| PASST + BeOH | 1.0 | 6.3 | 12.0 | 21.4 | 28.1 | 45.5 |
| PASST control | 1.0 | 4.0 | 6.9 | 12.1 | 16.1 | 26.7 |

TABLE 6
Low molecular species (dSEC clips) 4° C.

| sample | t = 0 | t = 4 w | t = 8 w | t = 12 w | t = 24 w |
|---|---|---|---|---|---|
| A45SuT | 1.6 | 1.3 | 1.4 | 2.3 | 2.5 |
| A52SuT | 1.2 | 1.6 | 1.3 | 1.7 | 1.7 |
| A58SuT | 1.4 | 1.7 | 1.5 | 1.4 | 1.4 |
| 50_SAST_100NaCl | 0.9 | 1.5 | 1.4 | 1.4 | 1.5 |
| 100_SAST_100NaCl | 1.0 | 1.5 | 1.7 | 2.0 | 2.6 |
| PASST + BeOH | 1.0 | 1.5 | 1.2 | 1.4 | 1.3 |
| PASST control | 1.2 | 1.4 | 1.4 | 1.7 | 1.6 |

TABLE 7
Low molecular species (dSEC clips) 25° C.

| sample | t = 0 | t = 4 w | t = 8 w | t = 12 w | t = 24 w |
|---|---|---|---|---|---|
| A45SuT | 1.6 | 3.6 | 5.9 | 8.4 | 13.1 |
| A52SuT | 1.2 | 2.9 | 2.9 | 5.8 | 9.2 |
| A58SuT | 1.4 | 2.2 | 2.8 | 4.0 | 5.7 |
| 50_SAST_100NaCl | 0.9 | 2.2 | 2.4 | 2.9 | 4.7 |
| 100_SAST_100NaCl | 1.0 | 2.9 | 3.2 | 4.4 | 6.9 |
| PASST + BeOH | 1.0 | 2.2 | 2.2 | 3.3 | 4.4 |
| PASST control | 1.2 | 1.6 | 2.1 | 3.2 | 4.8 |

TABLE 8
Low molecular species (dSEC clips) 40° C.

| | t = | t = | t = | t = | t = | t = |
|---|---|---|---|---|---|---|
| sample | 0 | 2 w | 4 w | 8 w | 12 w | 24 w |
| A45SuT | 1.6 | 9.7 | 16.7 | 28.3 | 38.1 | 55.0 |
| A52SuT | 1.2 | 6.0 | 11.3 | 18.6 | 26.0 | 39.6 |
| A58SuT | 1.4 | 4.0 | 7.7 | 12.2 | 18.2 | 27.5 |
| 50_SAST_100NaCl | 0.9 | 3.6 | 5.9 | 8.6 | 12.8 | 18.7 |
| 100_SAST_100NaCl | 1.0 | 3.5 | 5.7 | 9.6 | 12.9 | 19.4 |
| PASST + BeOH | 1.0 | 3.6 | 5.8 | 9.8 | 13.2 | 20.5 |
| PASST control | 1.2 | 3.3 | 5.9 | 10.0 | 12.8 | 20.2 |

Conclusions: During long term storage at 25° C. and 40° C., the lower pH formulations, A45SuT, A52SuT, and A58SuT, exhibited undesirable levels of low molecular weight degraded or clipped species when analyzed using denatured size exclusion chromatography. The high concentration formulation 100_SAST_100NaCl stored at 25° C. and 40° C. began to show an increase in high molecular weight aggregates when analyzed using size exclusion chromatography, but performed similarly to the current commercial formulation at 4° C. The PASST+BeOH (which is the current commercial formulation modified by the addition of 0.004% polysorbate 20 and 0.9% benzyl alcohol) performed similarly to the current commercial formulation at both 4° C. and 25° C. but experienced an increase in high molecular weight species by SE-HPLC at later time points when stored at the elevated temperature of 40° C. However, the 50_SAST_100NaCl formulation maintained levels of high and low molecular weight species that were comparable to the current commercial formulation at all temperatures, even in the absence of phosphate buffer.

### Example 2: Pain Study

This study was a single-center, randomized, single-blind, crossover design in which 48 healthy men and women received single SC injections of 6 solutions.

Test formulations (detailed below in Table 9) were administered by a trained healthcare professional in 6 unique sequences with 8 subjects randomized to each sequence. Injections were administered in each quadrant of the anterior abdominal wall and administered approximately 1 hour apart. Within 30 seconds after each injection, subjects assessed their level of injection pain using a 100 mm Visual Analog Scale (VAS). Adverse events were collected from the beginning of the first injection through 30 days after the first injection. Safety follow-up phone calls were conducted on day 2 (24 hours after the sixth injection) and day 31 (±2 days).

TABLE 9
Tested Formulations

| Solution | Description | Volume | Composition |
|---|---|---|---|
| A. | Negative pain | 1.0 | 10 mM sodium acetate, 9% (w/v) sucrose, |
| | control | | 0.004% (w/v) polysorbate 20, pH 5.2 |
| B. | Commercial | .098 | 25 mM sodium phosphate, 25 mM L- |
| | formulation placebo | | arginine, 100 mM sodium chloride, 1.0% |
| | | | (w/v) sucrose, pH 6.3 |
| C. | Commercial | 1.0 | 100 mM sodium chloride, 25 mM sodium |
| | formulation placebo | | phosphate, 25 mM L-arginine, 1.0% (w/v) |
| | with benzyl alcohol | | sucrose, 0.01% (w/v) polysorbate 20, 0.9% |
| | | | (w/v) benzyl alcohol |
| D. | Test formulation | 1.0 | 100 mM sodium chloride, 25 mM L- |
| | without sodium | | arginine, 1.0% (w/v) sucrose, 0.01% (w/v) |
| | phosphate | | polysorbate 20 |
| E. | Test formulation | 0.51 | 100 mM sodium chloride, 25 mM L- |
| | without sodium | | arginine, 1.0% (w/v) sucrose, 0.01% (w/v) |
| | phosphate | | polysorbate 20 |
| F. | Commercial | 0.98 | 50 mg/mL etanercept in a solution consisting |
| | formulation | | of 100 mM sodium chloride, 25 mM sodium |
| | etanercept 50 | | phosphate, 25 mM L-arginine 1% sucrose, |
| | mg/mL | | pH 6.3 |

Statistical Methods: All analyses were conducted on the safety analysis set, which consisted of all subjects who received at least one solution. To provide 93.4% power to detect a 15 mm difference between the solutions (α=0.05, 2 sided), a sample size of 48 subjects (8 per sequence) was selected. A difference of 13 to 16 mm on the VAS is considered to be clinically meaningful (Gallagher et al, 2002, Am. J. Em. Med. v20; i4: 287-290).

Summary statistics (mean, SD, standard error [SE], median, minimum, maximum) were calculated for VAS scores by solution. VAS scores were analyzed using an analysis of variance (ANOVA) model, which included sequence, solution, and period as independent variables, and subject within sequence as a random effect. No adjustment was made for multiple comparisons.

Mean differences in VAS score for the primary and secondary comparisons, corresponding 95% confidence intervals (95% CI), and p-values were provided.

TABLE 10
Summary of VAS Scores

| immediately | | | | | | |
|---|---|---|---|---|---|---|
| after injection | Soln A | Soln B | Soln C | Soln D | Soln E | Soln F |
| N | 48 | 48 | 48 | 48 | 48 | 48 |
| Mean | 19.6 | 53.6 | 28,.7 | 29.8 | 29.6 | 53.4 |
| SD | 18.0 | 27.9 | 23 5 | 26.4 | 24.3 | 32.4 |
| SE | 2.6 | 4.0 | 3.4 | 3.8 | 3.5 | 4.7 |
| Median | 13.0 | 59.0 | 24.0 | 21.5 | 21.0 | 49.0 |
| Min, Max | 0, 66 | 1, 98 | 1, 99 | 0, 94 | 1, 86 | 2, 100 |

Conclusions: Both Solution C (non-product specific placebo with benzyl alcohol) and Solution D (non-product specific placebo without sodium phosphate) had significantly lower mean VAS scores than Solution B (etanercept placebo; p<0.001), indicating relatively less injection site pain with these 2 solutions. No significant differences in mean VAS scores were found between Solutions C and D, between Solution B (etanercept placebo) and Solution F (active etanercept), or between different injection volumes (0.51 and 1.0 mL). Solution A (negative pain control) was associated with the least pain compared with all the other solutions. Seven subjects had 1 or more adverse events. All of the adverse events were CTCAE Grade 1 non-serious injection site reactions.

### Example 3: Long Term Stability Testing of Formulation Candidates

A long-term study was performed to monitor etanercept stability in several new formulation candidates at 50 mg/mL. The stability was assessed on 1 mL fills in 1 mL staked glass needle syringes using SE-HPLC, HIC HPLC, dSEC HPLC, and particulate matter (HIAC) after storage at 4° C., 25° C. and 40° C. Osmolality and protein concentration were tested at time zero only, and pH was tested at time zero and after 12 weeks of storage to confirm that there was no pH drift. The results of the study showed that the formulations tested remained similar to the current commercial formulation after 12 weeks at the accelerated temperature of 40° C. as well as 24 weeks at the recommended storage of 2-8° C. and at the accelerated temperature of 25° C.

TABLE 11
Formulation conditions at 50 mg/mL etanercept

| Formulation | | Other | |
|---|---|---|---|
| Name | Buffer | Excipients | pH |
| PASST (control) | 25 mM | 25 mM L-arginine, 100 mM | 6.3 |
| | phosphate | NaCl, 1% sucrose, 0.010% | |
| | | polysorbate 20 | |
| SAST_100NaCl | none none | 25 mM L-arginine, 100 mM | 6.3 |
| | | NaCl, 1% sucrose, 0.010% | |
| | | polysorbate 20 | |
| SAST_120NaCl | | 25 mM L-arginine, 120 mM | 6.3 |
| | | NaCl, 1% sucrose, 0.010% | |
| | | polysorbate 20 | |

Materials: Enbrel drug substance in PASS (25 mM phosphate buffer, 25 mM L-arginine, 100 mM NaCl, 1% sucrose) at 50 mg/mL was used for this study. The material was diafiltered into PASS and SAS_100NaCl (25 mM L-arginine, 100 mM NaCl, 1% sucrose) at 50 mg/mL and then ultrafiltered to ˜75 mg/mL. The 50 mg/mL formulations were prepared by diluting the post-UF/DF PASS and SAS material with the corresponding solution. The SAST_120NaCl was prepared by diluting the 75 mg/mL SAS material using a concentrated NaCl stock solution to achieve a final concentration of 120 mM NaCl. A 1% stock solution of polysorbate 20 was prepared fresh and spiked into all formulation to a final concentration of 0.010%. All formulations were manually filled into 1 mL long BD glass syringes to a volume of 1 mL and then stoppered using an ASPU vacuuming stoppering unit.

Methods: The pH was measured using a Mettler Toledo SevenEasy pH meter combined with a Mettler Inlab MicroProbe. Samples were warmed to room temperature prior to measurements. Protein concentration measurements using absorbance at 280 nM for all samples were performed at room temperature using the DropSense96 UV/Vis Lab Chip DS system. Each sample was measured neat with at least three replicates (3 μL each), including a formulation solution blank. Osmolality was measured using The Advanced Osmometer Model 3900. Each measurement was performed using 250 μL of sample and 290 mOsm osmolality standards were tested to ensure the system was operating properly. Size exclusion HPLC was run on an Agilent 1100 HPLC with Chromeleon 7.2 software. Hydrophobic interaction HPLC was run on an Agilent 1100 HPLC with Chromeleon 7.2 software at an absorbance of 215 nm. Denatured size exclusion HPLC was run on an Agilent 1100 HPLC with Chromeleon 7.2 software. Sub-visible particle analysis was performed using a HACH HIAC/Royco particle counter system equipped with an HRLD-150 laser and Pharm Spec software. All samples were diluted with PASS formulation buffer to 25 mg/mL. Samples were thoroughly mixed, uncapped and degassed for 2 hours at 75 torr prior to analysis. Four (4) sips of 1.0 mL each (no tare volume) were performed, with the first sip discarded and the remaining three sips averaged. Data for particle sizes 2, 5, 10, and 25 μm was collected at all time points. The results account for the dilution and are reported as cumulative counts per milliliter.

Results and Discussion: The pH of all formulations was measured at time zero and after twelve weeks at all temperatures. No trends were observed as a function of time or storage temperature. The measured pH values for all samples can be found in Table 12. No drift in pH was observed after 52 weeks of storage at 4° C. 24 weeks of storage at 25° C. or twelve weeks of storage at 40° C.; all samples met the acceptance criteria of +/−0.2 pH units from the target Ph of 6.3.

TABLE 12
Measured pH for samples

| | | t = 52 w | t = 24 w | t = 12 w |
|---|---|---|---|---|
| Formulation Acronym | t = 0 | 4° C. | 25° C. | 40° C. |
| PASST | 6.27 | 6.30 | 6.32 | 6.28 |
| SAST_100NaCl | 6.23 | 6.15 | 6.25 | 6.20 |
| SAST_120NaCl | 6.22 | 6.16 | 6.24 | 6.15 |

The protein concentration of all formulations was tested at time zero. The protein concentration results for all samples can be found in Table 13. All samples met the acceptance criteria.

TABLE 13
Protein Concentration measurements

| Formulation Acronym | t = 0 |
|---|---|
| PASST | 51.1 |
| SAST_100NaCl | 51.4 |
| SAST_120NaCl | 51.0 |

Osmolality was tested at time zero only. The osmolality results for all samples can be found in Table 14. All formulations were at their target osmolality. Due to differences in buffer and excipient levels, the osmolality was not expected to be the same across the various formulations.

TABLE 14
Osmolality measurements

| Formulation Acronym | t = 0 (measured) | theoretical |
|---|---|---|
| PASST | 314 | 313 |
| SAST_100NaCl | 262 | 263 |
| SAST_120NaCl | 299 | 300 |

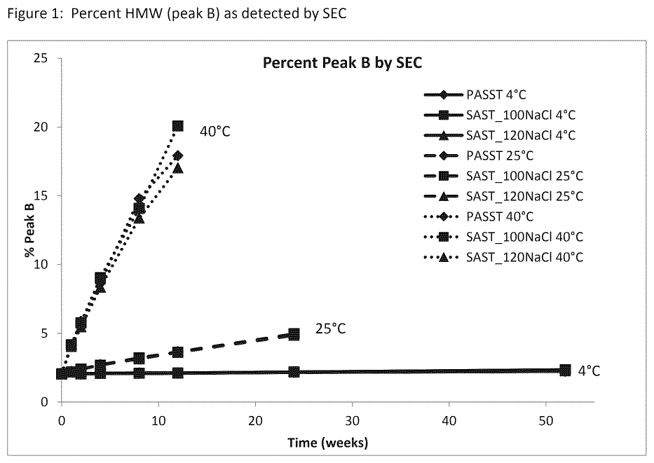

SE-HPLC was performed to monitor aggregation levels as a function of formulation condition, time and temperature. Peak B is the amount of high molecular weight species (aggregate) that forms. Results showed no differences in Peak B between the PASST control and the buffer-less formulations at 4° C. and 25° C., with minor differences being observed after twelve weeks at 40° C. (FIG. 1). Peak B represents the total aggregate detected by SE-HPLC for these formulations. All samples remained acceptable (Peak B≤6%,) after 52 weeks of storage at 4° C., 24 weeks of storage at 25° C., and after twelve weeks of storage at 40° C.

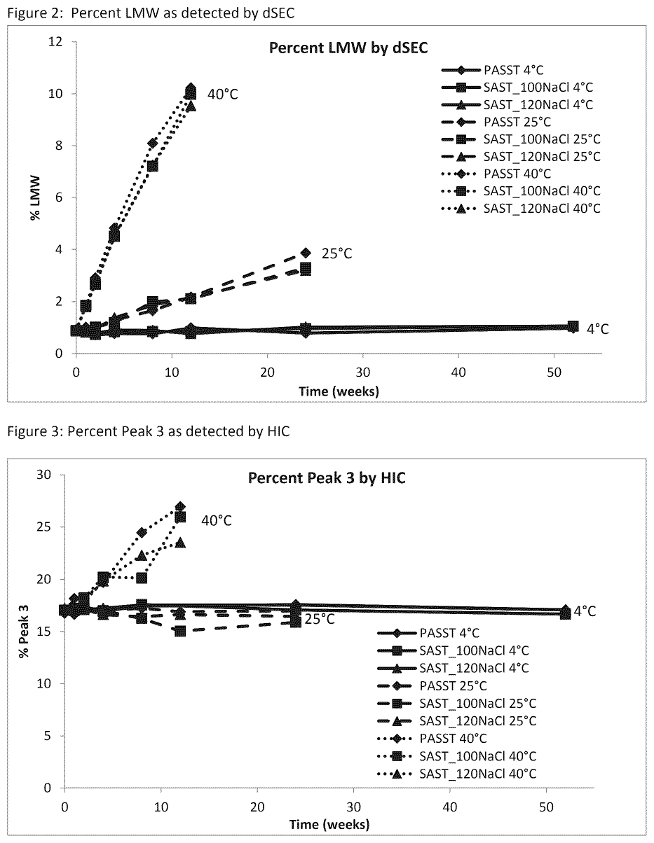

Denatured SE-HPLC was used to monitor clip species LMW. Results showed similar trends in HMW species, main peak, LMW between the formulations after 52 weeks (FIG. 2).

Changes in misfolded aggregates were monitored by HIC HPLC. Results at all tested temperatures showed no differences in Peak 3 between the PASST control and the bufferless formulations (FIG. 3). All samples remained within acceptable ranges (Peak 1≤5%, Peak 2≤70%, Peak 3≤28%) after 52 weeks of storage at 4° C., 24 weeks of storage at 25° C., and after twelve weeks of storage at 40° C.

Sub-visible particles were monitored by light obscuration particle counting (HIAC). Results were in line with historical PFS data and were similar between the formulations across all temperatures after twelve weeks. No trends could be established from this data set, as a single vial containing three pooled syringes was used at each time point and there is a high level of syringe-to-syringe variability in the contribution to silicone oil droplets.

Conclusions: The long-term stability of several new reformulation candidates and the current commercial formulation with the addition of polysorbate was assessed at 4° C., 25° C. and 40° C. No significant differences were observed between the formulations after 52 weeks at 4° C. and 24 weeks at 25° C. by SE-, dSEC, or HIC HPLC assays as well as by light obscuration; minor differences were observed after twelve weeks at 40° C. by HPLC assays. No drift in pH was observed and all formulations remained within acceptable ranges. The results of the study showed that the SAST_120NaCl and SAST 100NaCl formulations at 50 mg/mL were stable and similar to the current commercial formulation after twelve weeks at the recommended storage temperature of 2° C. to 8° C.

### Example 4: Freeze/Thaw and Long Term Stability of Top Reformulation Candidates in Stainless Steel Containers

A freeze/thaw cycling study was performed to monitor etanercept stability in three new formulations candidates at 50 mg/mL. Formulations compared to the current commercial formulation PASS (25 mM phosphate buffer, 25 mM L-arginine, 100 mM NaCl, 1% sucrose) were SAST_100NaCl (25 mM L-arginine, 100 mM NaCl, 1% sucrose, 0.010% polysorbate 20), SAS_120NaCl (25 mM L-arginine, 120 mM NaCl, 1% sucrose), and SAST_120NaCl (25 mM L-arginine, 120 mM NaCl, 1% sucrose, 0.010% polysorbate 20). The stability to aggregation when cycled between −30° C. and 4° C. in 55 mL stainless steel cryo vessels was assessed using SE-HPLC up to five freeze/thaw cycles.

Additionally, a long-term study was performed to monitor etanercept stability in a new formulation candidate at 50 mg/mL. Formulation compared to the current commercial formulation PASS (25 mM phosphate buffer, 25 mM L-arginine, 100 mM NaCl, 1% sucrose), was SAST_120NaCl (25 mM phosphate buffer, 25 mM L-arginine, 100 mM NaCl, 1% sucrose, 0.010% polysorbate 20). The stability when stored in 10 mL and 55 mL stainless steel cryo vessels was assessed using SE-HPLC, HIC HPLC, dSEC HPLC, and particulate matter (HIAC). Storage temperatures and time points were −30° C. for up to 36 months and 4° C. for up to twelve months. Results at 52 weeks are presented here.

Results: The pH of all formulations remained consistent at the 52 week time point and through five cycles of freeze/thaw.

TABLE 15
pH, Protein concentration and Osmolality

| | | | pH | | |
|---|---|---|---|---|---|
| | Conc. | Osmolality | | 52 wk | 52 wk |
| Sample | (mg/mL) | (mOsm) | t = 0 | −30° C. | 4° C. |
| PASS | 49.5 | 304 | 6.34 | 6.20 | 6.22 |
| SAST_120NaCl | 51.4 | 303 | 6.27 | 6.19 | 6.20 |

TABLE 16
pH, Protein Concentration and Osmolality for Freeze/Thaws

| | Conc. | Osmolality | pH | | |
|---|---|---|---|---|---|
| Sample | (mg/mL) | (mOsm) | 0 F/T | 3 F/T | 5 F/T |
| PASS | 47.9 | 310 | 6.30 | 6.29 | 6.27 |
| SAST_100NaCl | 48.8 | 259 | 6.19 | 6.18 | 6.16 |
| SAST_120NaCl | 48.1 | 294 | 6.17 | 6.19 | 6.18 |
| SAS_120NaCl | 48.6 | 300 | 6.17 | 6.17 | 6.18 |

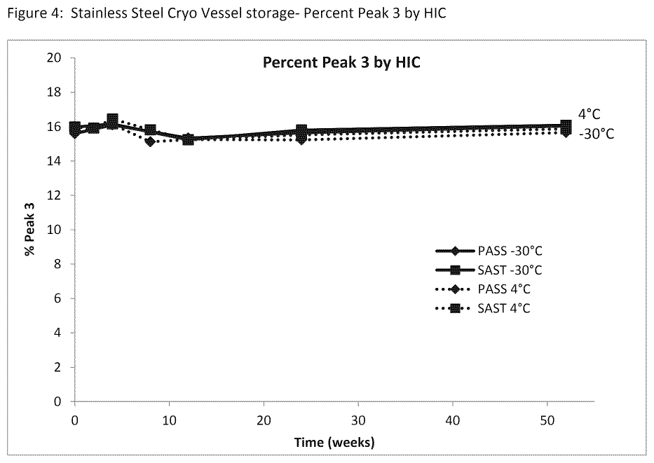

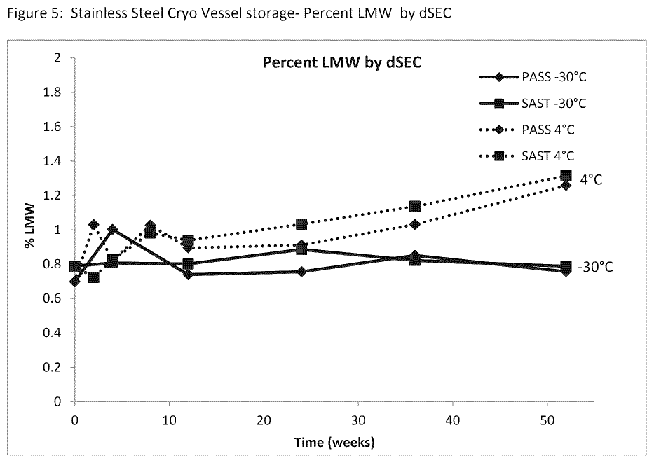

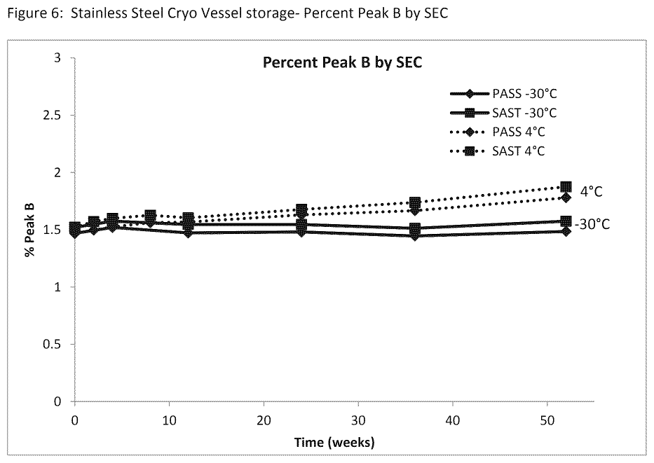

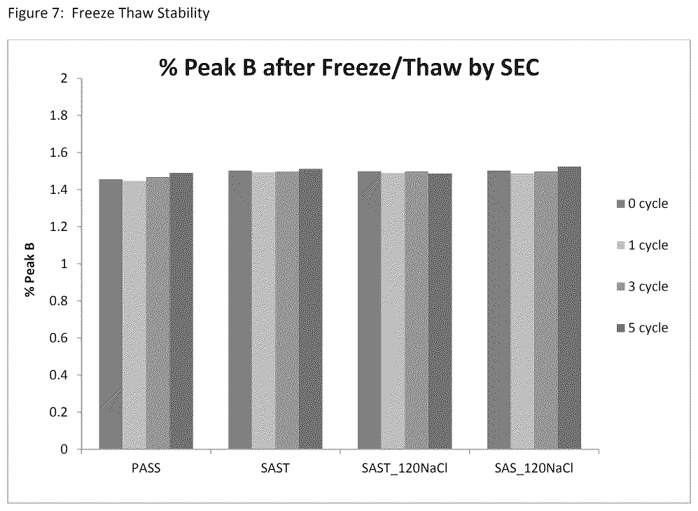

No trends were observed by HIAC for >10 um particles, although there were small increases in >2 and >5 um particles in the SAST_120NaCl formulation. As shown in FIG. 4, FIG. 5, and FIG. 6, no significant differences were observed between the formulations by HIC, dSEC, or SEC. No significant changes were observed by SEC between the formulations after exposure to five freeze thaw cycles. See FIG. 7.

Conclusion: The results of the study thus far showed that the new formulation tested remained similar to the current commercial formulation after 52 weeks storage in stainless steel cryo vessels at −30° C. as well as at 4° C.

### Example 5: Exchange into SAS and PASS Solutions

The purpose of these examples was to dialyze different preparations of etanercept in TMS (tris, mannitol, sucrose) into the test formulation (L-arginine, sucrose, NaCl) and compare the final pH to the target pH.

Materials: Etanercept: 25 mg/mL, in TMS (10 mM Tris HCl, 4% mannitol, 1% sucrose, pH 7.4); SAS_100NaCl solution (100 mM NaCl, 25 mM L-arginine HCl, 1% sucrose, pH 6.3) for dialysis; PASS buffer (25 mM Phosphate, 100 mM NaCl, 25 mM L-arginine HCl, 1% sucrose, pH 6.3); 10,000 MWCO centripreps; 3-12 mL Slide-A-Lyzer dialysis cassettes, 10,000 MWCO; a Mettler Toledo MP220 pH meter and Mettler Toledo InLab MicroProbe.

Methods: For the UF/DF example, 25 mg/mL etanercept in TMS was concentrated to

˜50 mg/mL by ultrafiltration using 30K MWCO Pellicon 3 cassettes on a Millipore Pellicon-2 mini system. The material was then diafiltered against SAS_100NaCl or PASS solution for 7 diavolumes, followed by concentration by ultrafiltration to 100 mg/mL. For the dialysis example, 25 mg/mL etanercept in TMS was concentrated to 50 mg/mL using 10,000 MWCO centripreps. The pH of the 50 mg/mL Sample in TMS was measured, as was the SAS dialysis solution, using the Mettler Toledo MP220 pH meter and InLab MicroProbe. The material was then dialyzed using 10,000 MWCO slide-a-lyzer dialysis cassettes. 9.5 mL of 50 mg/mL etanercept in TMS was added to the cassette and exchanged against 1000 mL of SAS100. Three exchanges were performed to achieve a 1,000,000 fold exchange. The first exchange occurred at 5 pm on Day 1 and went overnight. The second 1,000 mL exchange was at 8:30 am on Day 2. The third and final exchange was at 12:30 pm on Day 2. At 5:00 pm on Day 2 the protein was removed from the dialysis cassette (11 mL removed) and the pH was measured on the same Mettler Toledo MP220 pH meter. The measured pH was 6.98.

### Results

A summary of the results is shown below in Table 17.

TABLE 17
pH using various exchange methods and solutions

| | | | Pre- | | |
|---|---|---|---|---|---|
| Method | | Filtration | Method | Post | Number |
| of | | Solution | Exchange | Method | of |
| Exchange | Sample Name | pH | pH | pH | Exchanges |
| UF/DF | PASS, | 6.34 | 7.56 | 6.34 | 7 |
| | pH 6.3, | | | | Diafiltration |
| | 100 mg/mL | | | | volumes |
| UF/DF | SAS_100NaCl, | 6.38 | 7.56 | 6.98 | 7 |
| | pH 6.3, | | | | Diafiltration |
| | 100 mg/mL | | | | volumes |
| Dialysis | SAS_100NaCl, | 6.29 | 7.56 | 6.98 | 1,000,000 |
| | pH 6.3, | | | | fold |
| | 50 mg/mL | | | | exchange |

Conclusion: When the samples were ultrafiltrated/diafiltrated from a pre-exchange solution at pH 7.56 into the PASS buffer, the target pH of 6.34 was attained. However, when the samples were ultrafiltrated/diafiltrated into the SAS_100NaCl solution, the pH of the post-dialysis material that was achieved was 6.98, which was higher than expected and was not close to the final target pH of 6 3. Using dialysis as the exchange method into SAS_100NaCl achieved the same results.

### Example 6: UF/DF Pool

Introduction: The formulation solution that was chosen following this study was termed SAS (120 mM sodium chloride, 25 mM L-arginine, 1% sucrose, pH 6.3), without added phosphate buffer. Since the previous example demonstrated that it was difficult to attain the target pH of 6.3 when either dialyzing or using UF/DF when starting from etanercept in a sample at pH 7.56, a different method of exchange into the SAS formulation was needed. Two methods were evaluated that utilized separate final UF/DF starting material: 1) column 3 (AEX) intermediate pool as the starting material, and 2) Enbrel drug substance in PASS formulation buffer (PASS DS intermediate pool) as the starting material. Each method is described below and summarizes the development of a final UF/DF unit operation step to produce 50 g/L SAS formulated etanercept, including preparation of SAS formulation solution, final UF/DF load conditioning and processing.

Methods: The SAS formulation solution is composed of 120 mM sodium chloride, 25 mM L-arginine, 1% sucrose, pH 6.3. An SAS formulation solution was titrated to pH 6.3 using 10 N NaOH. The volume of titrant required to reach the specific pH range was 4.4 μL/L SAS formulation solution. During execution of the SAS final UF/DF unit operation, following equilibration of the membranes at 10 L/m² with the SAS formulation solution, the pH of the permeate remained close to the pH of WFI rather than the pH of the SAS formulation solution. Without being bound to a particular theory, this is believed to be due to the low buffering capacity of the SAS formulation solution. The expected range for conductivity of the permeate following membrane equilibration using the range of the SAS formulation solution preparation is 12-16 mS/cm. A higher post equilibration pH than that of the SAS formulation solution is expected and should not raise concern or indicate that the membranes are not equilibrated.

AEX Intermediate Pool Starting Material: Prior to transferring the AEX intermediate pool into the retentate tank of an UF/DF tank, the pool was conditioned using 2 M HCl to a target pH of 6.3 (acceptable range 6.2-6.4) The volume of titrant required to reach the specific pH range was approximately 2.8 mL/L AEX intermediate pool.

Eight examples performed during development of the SAS final UF/DF unit operation step, using AEX intermediate pool as the starting material, are listed in Table 18. Two parameters were investigated: the pH of the conditioned AEX intermediate pool and the pH of the SAS formulation solution. The first three runs were analyzed for pH, conductivity, osmolality, protein concentration, and product quality. Runs 4 through 7 were only measured for pH, conductivity, osmolality, and protein concentration in order to determine impact of formulation solution pH and load pH impact UF/DF pool pH.

TABLE 18
AEX Intermediate Pool Starting Material: Load,
Exchange Solution, and final UF/DF pool pH

| Run | Target | Target | UF/DF |
|---|---|---|---|
| Number | Load pH | SAS | Pool pH |
| 1 | 6.3 | 6.3 | 6.26 |
| 2 | 6.3 | 6.3 | 6.33 |
| 3 | 6.3 | 6.3 | 6.22 |
| 4 | 6.3 | 5.6 | 6.22 |
| 5 | 6.2 | 5.3 | 6.06 |
| 6 | 6.4 | 7.3 | 6.94 |
| 7 | 6.2 | 5.6 | 6.14 |
| 8 | 6.4 | 6.5 | 6.43 |

Results: The product quality results for the final SAS UF/DF pool, generated using AEX intermediate pool as the starting material, are shown in Table 19. The step yield for Run 1 was outside of the acceptance criteria; however, it was most likely an artifact of bench-scale processing and considered not significant to the conclusions of the study. All three final UF/DF SAS runs also met acceptance criteria for product quality using SEC and HIC analysis, as described above.

TABLE 19
AEX Intermediate Pool Starting Material:
Final SAS UF/DF Pool Product Quality

| | Acceptance | Final SAS UF/DF Pool | | |
|---|---|---|---|---|
| Parameter | Criteria | Run 1 | Run 2 | Run 3 |
| pH | 6.1-6.5 | 6.26 | 6.22 | 6.33 |
| Protein Concentration | 49-51 | 50.08 | 49.68 | 49.90 |
| (mg/mL) | | | | |
| Step Yield (%) | 95-103 | 93.4 | 99.5 | 100.5 |

### Conditioned AEX Intermediate Pool Stability

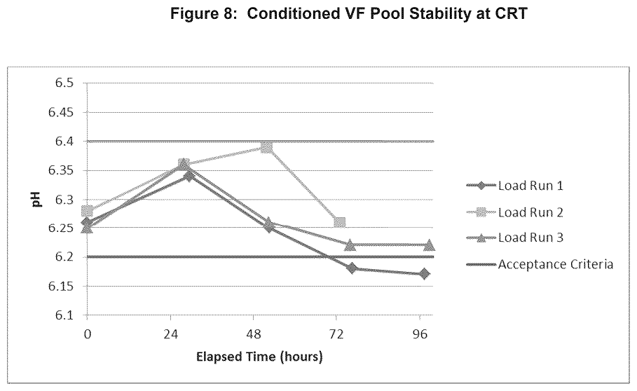

The conditioned AEX intermediate pool can be held for up to 52.6 hours at controlled room temperature (CRT). The pH of the pool during the hold is shown in FIG. 8.

### UF/DF Pool Stability

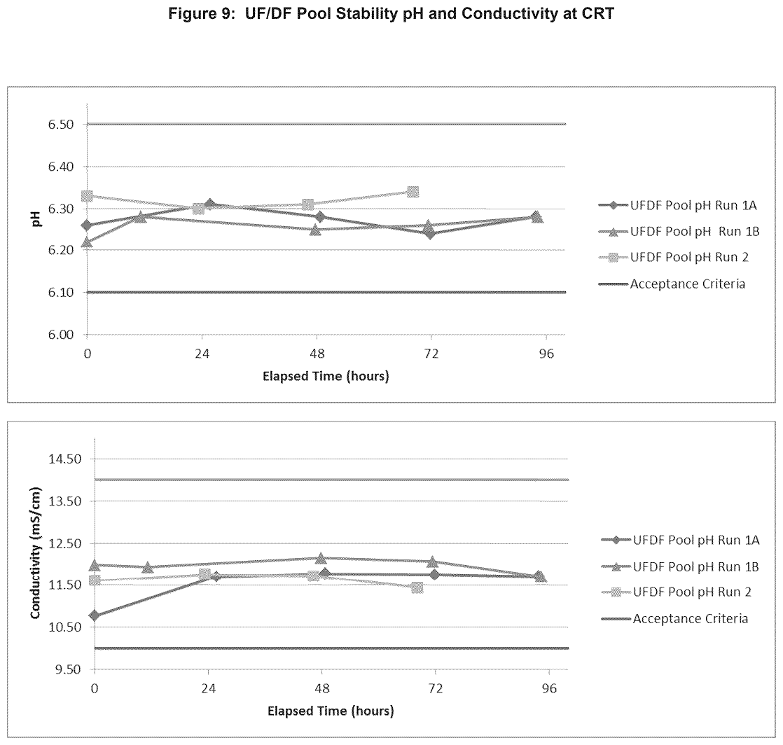

The final UF/DF SAS pool, generated using AEX intermediate pool as the starting material, can be held for up to 96.3 hours at CRT. The pH and conductivity during the hold are shown in FIG. 9A and B. Over the 96.3 hour hold, the pH and conductivity remain within acceptable limits.

PASS DS Intermediate Pool Starting Material: No conditioning is required prior to transferring the PASS DS intermediate pool into the UF/DF retentate tank because the PASS DS intermediate pool is already within the acceptable pH range. In addition, since the starting material is 50 mg/mL PASS formulated Enbrel DS, the pool does not need to be concentrated to 50 g/L because it is already at the correct concentration to perform diafiltration.

One example performed during development of the SAS final UF/DF unit operation step, to evaluate starting material source, is listed in Table 20. This example utilized DS PASS intermediate pool as the starting material and was analyzed for pH, conductivity, osmolality, protein concentration, and product quality.

TABLE 20
PASS DS Intermediate Pool Starting Material:
Load, Exchange Solution, and final UF/DF pool pH

| | Target | Targer SAS | UF/DF |
|---|---|---|---|
| Run Number | Load pH | Solution pH | Pool pH |
| 1 | 6.3 | 6.3 | 6.23 |

Results: The product quality results for the final SAS UF/DF pool, generated using PASS DS intermediate pool as the starting material, are shown in Table 21. The step yield for Run 1 was outside of the acceptance criteria; however, it was most likely an artifact of bench-scale processing and considered not significant to the conclusions of the study. The final SAS UF/DF pool also met acceptance criteria for product quality using SEC and HIC analysis, as described above.

TABLE 21
PASS DS Intermediate Pool Starting Material:
Final SAS UF/DF Pool Product Quality

| | Acceptance | Final SAS UF/DF Pool |
|---|---|---|
| Parameter | Criteria | Run 1 |
| pH | 6.1-6.5 | 6.23 |
| Protein Concentration (mg/mL) | 49-51 | 49.60 |
| Step Yield (%) | 95-103 | 105.7 |

### PASS DS Intermediate Pool Stability

The PASS pool does not require conditioning prior to UF/DF processing with SAS solution because this intermediate pool is already at the target pH (6.3). A pool hold study was not performed for this intermediate pool because the conditions of the pool were unchanged from Enbrel PASS DS. The pool can be held for up to 96 hours at 25° C.

### UF/DF Pool Stability

The final UF/DF SAS pool, generated using PASS DS intermediate pool as the starting material, can be held for up to 96.3 hours at CRT. The pH and conductivity during the hold are shown in FIG. 9A and B. Over the 96.3 hour hold, the pH and conductivity remain within acceptable limits.

### SAS Formulation Solution Stability

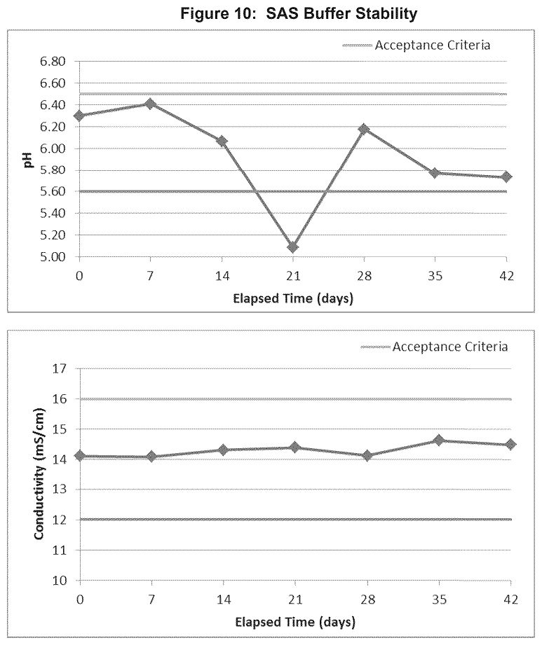

The SAS formulation solution can be held for up to 28 days at CRT. The pH and conductivity are shown in FIG. 10A and B. Over the 42 day hold in small scale stainless steel stability chambers with very small headspace, the SAS formulation solution is demonstrated to maintain a pH within 5.6 to 6.5. There was precipitation observed at the 35 day and 42 day time points. The 21 day time point measurement of 5.09 appears to be an outlier due to the fact that the subsequent time points are within the proposed acceptance criteria.

Conclusions: The final UF/DF unit operation can produce 50 g/L SAS formulation product and achieve consistent product quality compared to the current commercial PASS formulation product under the following process recommendations: 1) utilizing either AEX intermediate pool, or PASS DS intermediate pool, as the starting material 2) the SAS solution can be held for at least 28 days at CRT and maintain a pH of 5.6 to 6.5, 3) the conditioned AEX intermediate pool can be held at CRT for at least 52.6 hours and maintain a pH of 6.3±0.1, and 4) the SAS formulated UF/DF pool can be held at CRT for at least 96.3 hours and maintain a pH of 6.1 to 6.5 and a conductivity of 10 to 14 mS/cm.

### Example 7: Isotonic Alternative Formulations

The goal of this example was to determine the effect on aggregation of increased levels of arginine, sucrose or sodium chloride on etanercept stability at 75 mg/mL at 40° C. The levels of these excipients were each increased to maintain an isotonic formulation without added phosphate buffer. Additionally, histidine was evaluated as a buffer to replace phosphate. The formulations tested are summarized in Table 22.

TABLE 22
Formulations for isotonic alternative formulations
at pH 6.3 with 0.01% PS20

| | | L-Arginine | NaCl | Sucrose |
|---|---|---|---|---|
| Formulation Name | Buffer | (mM) | (mM) | (% w/w) |
| PASST | 25 mM phosphate | 25 | 100 | 1% |
| SAST_100NaCl | none | 25 | 100 | 1% |
| SAST_30Arg | none | 30 | 100 | 1% |
| SAST_35Arg | none | 35 | 100 | 1% |
| SAST_40Arg | none | 40 | 100 | 1% |
| SAST_2Suc | none | 25 | 100 | 2% |
| SAST_120NaCl | none | 25 | 120 | 1% |
| HASST | 10 mM histidine | 25 | 100 | 1% |

Materials: Enbrel drug substance in PASS (25 mM phosphate buffer, 25 mM L-arginine, 100 mM NaCl, 1% sucrose) at 50 mg/mL was used for this study. The material was dialyzed into the new formulations (without polysorbate) and concentrated to using 75 mg/mL using 30,000 MWCO centripreps. A 1% stock solution of polysorbate 20 was prepared fresh and spiked into all formulation to a final concentration of 0.01%. All formulations were manually filled into 1 mL long BD glass syringes to a volume of 1.0 mL and then stoppered using an ASPU vacuuming stoppering unit.

Methods: The pH was measured using a Mettler Toledo pH meter combined with a Mettler MicroProbe. Samples were warmed to room temperature prior to measurements. Osmolality was measured using The Advanced Osmometer Model 3900. Each measurement was performed using 250 μL of sample and 290 osmolality standards were tested to ensure the system was operating properly. Size exclusion HPLC was run on an Agilent 1100 HPLC with Chromeleon 7.2 software.

Results: The concentration, pH and osmolality are shown in Table 23. Aggregation rates at 75 mg/mL at 40° C. were similar to the commercial formulation composition for all
phosphate-free formulations with increased levels of L-arginine, sucrose and NaCl, as shown in Table 24. Additionally, use of histidine instead of phosphate as a buffer led to increased aggregation rates at 40° C.

TABLE 23
Concentration, pH and osmolality for
isotonic alternative formulations

| | Conc | | |
|---|---|---|---|
| Sample | (mg/mL) | pH | osmolality |
| PASST | 76.3 | 6.27 | 308 |
| SAST_100NaCl | 76.8 | 6.30 | 266 |
| SAST_30Arg | 74.9 | 6.28 | 271 |
| SAST 35Arg | 76.9 | 6.27 | 280 |
| SAST_40Arg | 76.5 | 6.26 | 288 |
| SAST_2Suc | 75.2 | 6.26 | 291 |
| SAST_120NaCl | 77.1 | 6.24 | 301 |
| HASST | 76.3 | 6.36 | 272 |

TABLE 24
SEC aggregate/HMW levels, % of total, 40° C.

| | 0 | 1 | 2 | 4 | 8 | 12 |
|---|---|---|---|---|---|---|
| Sample | Week | Week | Week | Week | Week | Week |
| PASST | 2.3 | 5.3 | 6.6 | 12.0 | 16.1 | 26.1 |
| SAST_100NaCl | 2.3 | 5.0 | 6.4 | 11.0 | 15.3 | 24.1 |
| SAST_30Arg | 2.4 | 5.1 | 6.6 | 11.3 | 15.9 | 24.8 |
| SAST_35Arg | 2.4 | 5.2 | 6.8 | 11.5 | 16.7 | 25.3 |
| SAST_40Arg | 2.4 | 5.1 | 6.7 | 11.0 | 16.1 | 23.7 |
| SAST_2Suc | 2.4 | 4.9 | 6.6 | 10.7 | 16.2 | 23.4 |
| SAST_120NaCl | 2.4 | 5.0 | 5.9 | 11.0 | 16.7 | 23.7 |
| HASST | 2.3 | 6.2 | 9.1 | 14.8 | 22.9 | 32.3 |

### Example 8: Stability of Formulations With Various Levels of Polysorbate 20

A long-term study was performed to monitor etanercept stability at 0, 0.005, 0.01 and 0.015% polysorbate 20 in the SAS formulation at 50 mg/mL etanercept. In addition, a high concentration formulation of SAST at 100 mg/mL etanercept was tested. The stability was assessed on 1 mL fills in 1 mL staked glass needle syringes using SE-HPLC, dSEC HPLC, and particulate matter (HIAC) after storage at 4° C., 25° C. and 40° C. Osmolality, pH and protein concentration were tested at time zero only. The results of the study showed that the 50 mg/mL formulations tested remained similar to the current commercial formulation after 24 weeks at recommended storage of 2-8° C., as well as at accelerated temperatures of 25° C. and 40° C. The 100 mg/mL SAST formulation performed comparably to the 50 mg/mL formulations in terms of pH and subvisible particles; differences in aggregate levels by SEC were attributed to protein concentration.

TABLE 25
Formulation conditions at 50 mg/mL etanercept

| | | | | Protein |
|---|---|---|---|---|
| Formulation | | | Poly- | Conc |
| Name | Buffer | Other Excipients | sorbate | (mg/mL) |
| PASS | 25 mM | 25 mM L-arginine, | 0 | 50 |
| | phosphate | 100 mM NaCl, | | |
| | | 1% sucrose | | |
| SAS000T | None | 25 mM L-arginine, | 0 | 50 |
| | | 120 mM NaCl, | | |
| | | 1% sucrose | | |
| SAS005T | None | 25 mM L-arginine, | 0.005 | 50 |
| | | 120 mM NaCl, | | |
| | | 1% sucrose | | |
| SAS010T | None | 25 mM L-arginine, | 0.01 | 50 |
| | | 120 mM NaCl, | | |
| | | 1% sucrose | | |
| SAS015T | None | 25 mM L-arginine, | 0.015 | 50 |
| | | 120 mM NaCl, | | |
| | | 1% sucrose | | |
| 100_SAS010T | None | 25 mM L-arginine, | 0.010 | 100 |
| | | 120 mM NaCl, | | |
| | | 1% sucrose | | |

Materials: Enbrel drug substance in TMS (10 mM tris buffer, 4% mannitol, 1% sucrose) at 25 mg/mL was used for this study. The bulk used for the SAS formulation was titrated to pH 6.3. The material was ultrafiltered to ˜50 mg/mL etanercept, then diafiltered into PASS (25 mM phosphate, 25 mM L-arginine, 120 mM NaCl, 1% sucrose) or SAS (25 mM L-arginine, 120 mM NaCl, 1% sucrose) at 50 mg/mL etanercept. The material for the high concentration arm was then ultrafiltered to 100 mg/mL etanercept. A 1% stock solution of polysorbate 20 was prepared fresh and spiked into the formulations to the final concentrations as listed in Table 25. All formulations were manually filled into 1 mL long BD glass syringes to a volume of 1 mL and then stoppered using an ASPU vacuuming stoppering unit.

Results and Discussion: The pH of all formulations was measured at time zero and after twelve weeks at 40° C. and 24 weeks at 4° C. and 25° C. No trends were observed as a function of time or storage temperature. The measured pH values for all samples can be found in Table 26. No drift in pH was observed after twelve weeks of storage at 40° C. or 24 weeks at 4° C. and 25° C. and all samples met the acceptance criteria of +/−0.2 pH units from the target pH of 6.3. The protein concentration and osmolality of all formulations was tested at time zero. The protein concentration and osmolality results for all samples can be found in Table 26.

TABLE 26
Concentration, osmolality and pH

| | | | | pH | pH | pH |
|---|---|---|---|---|---|---|
| | Conc | Osmolality | pH | 24 wk | 24 wk | 12 wk |
| Sample | (mg/mL) | (mOsm) | t = 0 | 4° C. | 25° C. | 40° C. |
| PASS | 51.6 | 318 | 6.32 | 6.34 | 6.34 | 6.33 |
| SAS000T | 52.1 | 306 | 6.33 | 6.31 | 6.33 | 6.37 |
| SAS005T | 51.2 | 304 | 6.33 | 6.30 | 6.30 | 6.32 |
| SAS010T | 51.5 | 304 | 6.34 | 6.30 | 6.30 | 6.36 |
| SAS015T | 51.5 | 301 | 6.30 | 6.30 | 6.30 | 6.35 |
| 100_SAS010T | 102.8 | 304 | 6.32 | 6.28 | 6.29 | 6.36 |

SE-HPLC was performed to monitor aggregation levels as a function of formulation condition, time and temperature. Peak B is the amount of high molecular weight species (aggregate) that forms. Results showed no differences in Peak B between the PASS control and the bufferless formulations at all temperatures at their respective protein concentrations (Table 27-29). Peak B represents the total aggregate detected by SE-HPLC for these formulations. All 50 mg/mL samples remained acceptable (Peak B≤6%,) after 24 weeks of storage at 4° C. and 25° C., and after 2 weeks of storage at 40° C.

Sub-visible particles were monitored by light obscuration particle counting (HIAC). Results were in line with historical PFS data and were similar between the formulations across all temperatures after 24 weeks (Table 30).

TABLE 27
SEC analysis of Peak B, % of total, 4° C.

| sample | t = 0 wk | t = 4 wk | t = 8 wk | t = 12 wk | t = 24 wk |
|---|---|---|---|---|---|
| PASS | 1.7 | 1.7 | 1.7 | 1.7 | 1.8 |
| SAS000T | 1.7 | 1.8 | 1.8 | 1.8 | 1.9 |
| SAS005T | 1.7 | 1.8 | 1.8 | 1.8 | 1.9 |
| SAS010T | 1.7 | 1.8 | 1.8 | 1.9 | 1.9 |
| SAS015T | 1.7 | 1.8 | 1.8 | 1.8 | 1.9 |
| 100_SAS010T | 1.9 | 2.0 | 2.1 | 2.2 | 2.4 |

TABLE 28
SEC analysis of Peak B, % of total, 25° C.

| | t = | t = | t = | t = | t = | t = |
|---|---|---|---|---|---|---|
| sample | 0 wk | 2 wk | 4 wk | 8 wk | 12 wk | 24 wk |
| PASS | 1.7 | 2.1 | 2.4 | 2.9 | 3.5 | 4.8 |
| SAS000T | 1.7 | 2.1 | 2.5 | 2.9 | 3.5 | 5.0 |
| SAS005T | 1.7 | 2.2 | 2.5 | 2.9 | 3.5 | 4.9 |
| SAS010T | 1.7 | 2.1 | 2.5 | 3.0 | 3.6 | 4.8 |
| SAS015T | 1.7 | 2.2 | 2.4 | 3.0 | 3.5 | 4.8 |
| 100_SAS010T | 1.9 | 2.8 | 3.4 | 4.4 | 5.4 | 7.6 |

TABLE 29
SEC analysis of Peak B, % of total, 40° C.

| sample | t = 0 wk | t = 2 wk | t = 4 wk | t = 8 wk | t = 12 wk |
|---|---|---|---|---|---|
| PASS | 1.7 | 5.7 | 10.4 | 17.4 | 20.6 |
| SAS000T | 1.7 | 4.9 | 8.9 | 15.4 | 19.2 |
| SAS005T | 1.7 | 5.2 | 9.1 | 15.9 | 19.4 |
| SAS010T | 1.7 | 5.2 | 9.3 | 15.8 | 19.2 |
| SAS015T | 1.7 | 5.4 | 9.4 | 15.9 | 19.0 |
| 100_SAS010T | 1.9 | 9.0 | 15.6 | 24.5 | 27.1 |

TABLE 30
Particles/mL (light obscuration), 4° C.

| | 2 μm | | 10 μm | | 25 μm | |
|---|---|---|---|---|---|---|
| | t = | t = | t = | t = | t = | t = |
| | 0 wk | 24 wk | 0 wk | 24 wk | 0 wk | 24 wk |
| PASS | 19197 | 3422 | 1785 | 670 | 25 | 67 |
| SAS000T | 13004 | 4489 | 1265 | 762 | 60 | 104 |
| SAS005T | 12344 | 1292 | 502 | 59 | 7 | 4 |
| SAS010T | 11964 | 2254 | 1058 | 167 | 9 | 2 |
| SAS015T | 11388 | 7283 | 934 | 591 | 2 | 4 |
| 100_SAS010T | 58087 | 8931 | 559 | 816 | 3 | 0 |

Conclusions: The long-term stability of a reformulation candidate at 50 mg/mL etanercept with levels of polysorbate from 0 to 0.015% and the current commercial formulation was assessed at 4° C., 25° C. and 40° C.; a high concentration arm at 100 mg/mL etanercept was also tested for the SAS010T formulations. No significant differences were observed between the formulations at their respective protein concentrations after 24 weeks by SE-, dSEC, or HIC HPLC assays as well as by light obscuration. No drift in pH was observed and all formulations remained within acceptable ranges. The results of the study showed that the SAST_120NaCl formulations at 50 mg/mL were stable and similar to the current commercial formulation after 24 weeks at the recommended storage temperature of 2° C. to 8° C.

### Example 9: Stability of Formulations in Plastic Syringes

A long-term study was performed to monitor etanercept stability in the PASS and SAS formulations at 50 mg/mL etanercept in COP plastic silicone oil free pre-filled syringe systems compared to glass siliconized pre-filled syringes. The stability was assessed on 1 mL fills in the various syringe systems using SE-HPLC, pH and particulate matter (HIAC) after storage at 4° C., 25° C. and 40° C. Protein concentration was tested at time zero only.

Materials: Etanercept drug substance in TMS (10 mM tris buffer, 4% mannitol, 1% sucrose) at 25 mg/mL etanercept was used for this study. The bulk used for the SAS formulation was titrated to pH 6.3. The material was ultrafiltered to ˜50 mg/mL etanercept, then diafiltered into PASS (25 mM phosphate, 25 mM L-arginine, 120 mM NaCl, 1% sucrose) or SAS (25 mM L-arginine, 120 mM NaCl, 1% sucrose) at 50 mg/mL etanercept. All formulations were manually filled into 1 mL long glass syringes or 1 mL COP plastic silicone oil free syringes (COP_A and COP_B) to a volume of 1 mL and then stoppered using a vacuuming stoppering unit.

Methods: The pH was measured using a Mettler Toledo SevenEasy pH meter combined with a Mettler Inlab MicroProbe. Samples were warmed to room temperature prior to measurements. Protein concentration measurements using absorbance at 280 nM for all samples were performed at room temperature using the Nano Drop system. Size exclusion HPLC was run on an Agilent 1100 HPLC with Chromeleon 7.2 software. Sub-visible particle analysis was performed using a HACH HIAC/Royco particle counter system equipped with an HRLD-150 laser and Pharm Spec software. All samples were diluted with PASS formulation buffer to 25 mg/mL. Samples were thoroughly mixed, uncapped and degassed for 2 hours at 75 torr prior to analysis. Four (4) sips of 1.0 mL each (no tare volume) were performed, with the first sip discarded and the remaining 3 sips averaged. Data for particle sizes 2, 5, 10, and 25 um was collected at all timepoints. The results account for the dilution and are reported as cumulative counts per mL.

Results & Discussion: Stability in the plastic silicone oil free syringes is similar to stability in glass siliconized syringes. The protein concentration of all formulations was tested at time zero. The pH of all formulations was measured at time zero and after twelve weeks at 40° C. and 24 weeks at 4° C. and 25° C. No trends were observed as a function of time or storage temperature and all samples met the pH acceptance criteria of +/−0.2 pH units from the target pH of 6.3. The protein concentration and measured pH values for all samples can be found in Table 31.

TABLE 31
Protein concentration and pH results

| | | | pH | pH | pH |
|---|---|---|---|---|---|
| | Conc | pH | 24 wk | 24 wk | 12 wk |
| Sample | (mg/mL) | t = 0 | 4° C. | 25° C. | 40° C. |
| PASS_Glass | 50.9 | 6.3 | 6.4 | 6.4 | 6.4 |
| SAS_Glass | 52.3 | 6.3 | 6.4 | 6.4 | 6.3 |
| PASS_COP_A | 51.1 | 6.3 | 6.4 | 6.4 | 6.3 |
| SAS_COP_A | 51.9 | 6.3 | 6.4 | 6.4 | 6.3 |
| PASS_COP_B | 51.1 | 6.4 | 6.4 | 6.4 | 6.3 |
| SAS_COP_B | 52.2 | 6.3 | 6.4 | 6.4 | 6.3 |

SE-HPLC was performed to monitor aggregation levels as a function of formulation condition, time and temperature. Peak B is the amount of high molecular weight species (aggregate) that forms. Results showed no differences in Peak B between the glass syringes and the COP plastic silicone oil free syringes (Table 32-34). Peak B represents the total aggregate detected by SE-HPLC for these formulations. All samples remained acceptable (Peak B≤6%,) after 24 weeks of storage at 4° C. and 25° C.

TABLE 32
SEC analysis of Peak B, % of total, 4° C.

| sample | t = 0 wk | t = 4 wk | t = 8 wk | t = 12 wk | t = 24 wk |
|---|---|---|---|---|---|
| PASS_Glass | 2.9 | 3.0 | 3.1 | 3.1 | 3.1 |
| SAS_Glass | 3.0 | 3.1 | 3.2 | 3.2 | 3.1 |
| PASS_COP_A | 2.9 | 3.0 | 3.1 | 3.1 | 3.1 |
| SAS_COP_A | 3.0 | 3.1 | 3.2 | 3.2 | 3.2 |
| PASS_COP_B | 3.0 | 3.1 | 3.2 | 3.2 | 3.3 |
| SAS_COP_B | 3.0 | 3.1 | 3.2 | 3.2 | 3.2 |

TABLE 33
SEC analysis of Peak B, % of total, 25° C.

| | t = | t = | t = | t = | t = | t = |
|---|---|---|---|---|---|---|
| sample | 0 wk | 2 wk | 4 wk | 8 wk | 12 wk | 24 wk |
| PASS_Glass | 2.9 | 3.0 | 3.4 | 3.9 | 4.4 | 5.5 |
| SAS_Glass | 3.0 | 3.1 | 3.6 | 4.1 | 4.5 | 5.6 |
| PASS_COP_A | 2.9 | 3.1 | 3.4 | 3.9 | 4.4 | 5.4 |
| SAS_COP_A | 3.0 | 3.2 | 3.5 | 4.1 | 4.6 | 5.8 |
| PASS_COP_B | 3.0 | 3.2 | 3.6 | 4.1 | 4.6 | 5.5 |
| SAS_COP_B | 3.0 | 3.1 | 3.5 | 4.0 | 4.5 | 5.7 |

TABLE 34
SEC analysis of Peak B, % of total, 40° C.

| sample | t = 0 wk | t = 2 wk | t = 4 wk | t = 8 wk | t = 12 wk |
|---|---|---|---|---|---|
| PASS_Glass | 2.9 | 6.4 | 11.1 | 17.2 | 23.0 |
| SAS_Glass | 3.0 | 6.1 | 9.8 | 15.4 | 21.0 |
| PASS_COP_A | 2.9 | 6.5 | 9.9 | 16.2 | 21.4 |
| SAS_COP_A | 3.0 | 6.3 | 9.9 | 14.9 | 18.6 |
| PASS_COP_B | 3.0 | 6.4 | 10.7 | 17.8 | 19.6 |
| SAS_COP_B | 3.0 | 6.2 | 10.0 | 17.1 | 22.4 |

Sub-visible particles were monitored by light obscuration particle counting (HIAC) Results for formulations filled in BD glass syringes were in line with historical PFS data while subvisible particles are reduced in the silicone-oil free plastic syringes (Table 35).

TABLE 35
Particles/mL (light obscuration), 4° C.

| | 2 μm | | 10 μm | | 25 μm | |
|---|---|---|---|---|---|---|
| | t = | t = | t = | t = | t = | t = |
| | 0 wk | 24 wk | 0 wk | 24 wk | 0 wk | 24 wk |
| PASS_Glass | 10090 | 15994 | 468 | 726 | 14 | 2 |
| SAS_Glass | 12240 | 13340 | 1183 | 505 | 18 | 5 |
| PASS_COP_A | 93 | 130 | 2 | 6 | 0 | 0 |
| SAS_COP_A | 30 | 74 | 2 | 2 | 0 | 0 |
| PASS_COP_B | 81 | 204 | 3 | 4 | 0 | 2 |
| SAS_COP_B | 62 | 237 | 2 | 8 | 0 | 1 |

Conclusions: The long-term stability of the SAS formulation at 50 mg/mL etanercept and the current commercial etanercept formulation stored in glass siliconized syringes and COP silicone oil free syringes was assessed at 4° C., 25° C. and 40° C. No significant differences were observed between the formulations as a function of syringe type after 24 weeks by SE-HPLC. No drift in pH was observed and all formulations remained within acceptable ranges. Sub-visible particles were reduced in the COP silicone oil free plastic syringes and were consistent with historical PFS results when stored in the glass syringes. The results of the study showed that the SAS formulation at 50 mg/mL etanercept was stable and similar to the current commercial formulation after 24 weeks at the recommended storage temperature of 2° C. to 8° C. in various syringe types.

What is claimed is:

1. A pharmaceutical composition comprising between 75 mM and 150 mM NaCl, between 5 mM and 100 mM arginine, between 0.5% and 2% (w/v) sucrose, and between 40 mg/mL and 100 mg/mL etanercept, wherein the pharmaceutical composition comprises less than 2.0 mM total additional buffering agent, and the pH of the composition is between 6.1 and 6.5.

2. The pharmaceutical composition of claim 1, wherein the pharmaceutical composition comprises less than 1.5 mM total additional buffering agent.

3. The pharmaceutical composition of claim 2, wherein the pharmaceutical composition comprises less than 1.0 mM total additional buffering agent.

4. The pharmaceutical composition of claim 2, wherein the pharmaceutical composition comprises less than 0.5 mM total additional buffering agent.

5. The pharmaceutical composition of claim 2, wherein the pharmaceutical formulation comprises less than 0.25 mM total additional buffering agent.

6. The pharmaceutical composition of claim 2, wherein the pharmaceutical composition comprises 0.1 mM or less total additional buffering agent.

7. The pharmaceutical composition of claim 1, wherein the arginine is L-arginine.

8. The pharmaceutical composition of claim 7, wherein the L-arginine is L-arginine hydrochloride.

9. The pharmaceutical composition of claim 7, wherein the L-arginine is L-arginine base.

10. The pharmaceutical composition of claim 1, wherein the pharmaceutical composition maintains a pH between 6.1 and 6.5 when stored at controlled room temperature (CRT) for 2 weeks.

11. The pharmaceutical composition of claim 10, wherein the pharmaceutical composition maintains a pH between about 6.2 and about 6.3 when stored at controlled room temperature (CRT) for 2 weeks.

12. The pharmaceutical composition of claim 1, wherein the pharmaceutical composition maintains a pH of between 5.8 and 6.7 for at least two weeks when stored at approximately 25° C., and wherein less than 6% of the total etanercept is aggregated in a high molecular weight form as assessed using size exclusion chromatography.

13. The pharmaceutical composition of claim 1, wherein said pharmaceutical composition maintains a pH of between about 6.1 and about 6.5 for at least two weeks during said storage at approximately 25° C.

14. The pharmaceutical composition of claim 1, wherein said pharmaceutical composition maintains a pH of between about 6.2 and about 6.4 for at least two weeks during said storage at approximately 25≅ C.

15. The pharmaceutical composition of claim 1, wherein less than 28% of the total amount of etanercept is in a misfolded form as assessed using hydrophobic interaction chromatography after two weeks' storage at approximately 25° C.

16. The pharmaceutical composition of claim 1, wherein the pharmaceutical composition bas an osmolality of about 180 to about 420 milliosmolals.

17. The pharmaceutical composition of claim 16, wherein the pharmaceutical composition has an osmolality of about 250 to about 350 milliosmolals.

18. The pharmaceutical composition of claim 17, wherein the pharmaceutical composition has an osmolality of about 290 to about 310 milliosmolals.

19. The pharmaceutical composition of claim 18, wherein the pharmaceutical composition has an osmolality of about 300 to about 310 milliosmolals.

20. The pharmaceutical composition of claim 1 consisting essentially of about 50 mg/mL etanercept, about 120 mM NaCl, about 25 mM L-arginine hydrochloride, about 1% (w/v) sucrose, and water.

21. The pharmaceutical composition of claim 1 consisting of about 50 mg/mL etanercept, about 120 mM NaCl, about 25 mM L-arginine hydrochloride, about 1% (w/v) sucrose, and water.

22. The pharmaceutical composition of claim 1, further comprising polysorbate 20.

23. The pharmaceutical composition of claim 22, wherein the polysorbate 20 concentration (w/v) is between about 0.001% and about 0.1%.

24. The pharmaceutical composition of claim 23, wherein the polysorbate 20 concentration (w/v) is about 0.005%, about 0.01%, or about 0.015%.

25. A method of making a pharmaceutical composition of etanercept that does not comprise an additional buffering agent and has a pH from 6.1 to 6.5, comprising exchanging an etanercept formulation comprising an additional buffering agent against a solution without any additional buffering agent, wherein the resulting pharmaceutical composition comprises between 40 mg/mL and 100 mg/mL etanercept, and wherein the etanercept formulation comprising an additional buffering agent and the solution without any additional buffering agent each has a pH from 6.1 to 6.5.

26. The method of claim 25 wherein the exchange step uses diafiltration.

27. The method of claim 25, wherein the solution without any additional buffering agent is isotonic.

28. The method of claim 25, wherein the solution without any additional buffering agent contains sucrose, arginine, and NaCl.

29. The method of claim 28, wherein the solution without any additional buffering agent contains between 75 mM and 150 mM NaCl; between 5 mM and 100 mM arginine; and between 0.5% and 2% (w/v) sucrose.

30. The method of claim 28, wherein the solution without any additional buffering agent consists essentially of about 120 mM NaCl, about 25 mM arginine, about 1% sucrose, and water.

31. The method of claim 25, further comprising the step of filtering the pharmaceutical composition.

32. The method of claim 25, further comprising the step of aliquoting the pharmaceutical composition into a drug product form.

33. The method of claim 25, wherein the etanercept formulation comprising an additional buffering agent contains between 75 mM and 150 mM NaCl, the solution without any additional buffering agent contains between 75 mM and 150 mM NaCl, and the pharmaceutical composition contains between 75 mM and 150 mM NaCl, and wherein the method does not comprise a NaCl removal step.

34. The method of claim 33, wherein the etanercept formulation comprising an additional buffering agent contains about 120 mM NaCl, the solution without any additional buffering agent contains about 120 mM NaCl, and the pharmaceutical composition contains about 120 mM NaCl.

35. The method of claim 25, wherein the method does not comprise a salt removal step.

36. A pharmaceutical composition comprising between 75 mM and 150 mM NaCl, between 5 mM and 100 mM arginine, between 0.5% and 2% (w/v) sucrose, and between 40 mg/mL and 100 mg/mL etanercept, wherein the pharmaceutical composition comprises less than 2.0 mM total additional buffering agent, and the pH of the composition is between 6.1 and 6.5, and wherein the pharmaceutical composition is made using the method of claim 25.

37. A kit comprising the pharmaceutical composition of claim 1 in a drug product form and instructions for storage and use.

38. A single-dose container containing the pharmaceutical composition of claim 1.

39. The single-dose container of claim 38, wherein said single dose container is a vial, a syringe, or an autoinjector.

40. The single-dose container of claim 38, containing an aqueous formulation consisting of etanercept at 50.0 mg/mL, sodium chloride at 120 mM, L-arginine hydrochloride at 25 mM, and sucrose at 1.0% (w/v).

41. A method of preparing a single-dose container, comprising filling the single-dose container with about a single dose of the pharmaceutical composition of claim 1 under sterile conditions.

42. A method of treating a patient having rheumatoid arthritis, psoriatic arthritis, ankylosing spondylitis, or psoriasis, comprising administering to said patient a pharmaceutical formulation of claim 1.
